(19) United States
(12) Patent Application Publication
(10) Pub. No.: US 2019/0054051 A1
(43) Pub. Date: Feb. 21, 2019

(54) DOPA Formulations for Treatments of Parkinson's Disease

(71) Applicant: The Board of Trustees of the Leland Stanford Junior University, Stanford, CA (US)

(72) Inventors: Mohamed Ahmed, San Jose, CA (US); Jayakumar Rajadas, Palo Alto, CA (US); Mohammed Inayathullah Nazir Ahmed, Santa Clara, CA (US); Wenchao Sun, Palo Alto, CA (US)

(73) Assignee: The Board of Trustees of the Leland Stanford Junior University, Stanford, CA (US)

(21) Appl. No.: 16/107,933

(22) Filed: Aug. 21, 2018

Related U.S. Application Data
(60) Provisional application No. 62/548,303, filed on Aug. 21, 2017.

(51) Int. Cl. A61K 31/198; A61P 25/14; A61K 9/20; A61K 9/48

(52) U.S. Cl. CPC A61K 31/198; A61P 25/14; A61K 31/341; A61K 9/4866; A61K 2300/00; A61K 9/2063

(57) ABSTRACT

The present disclosure provides compositions and methods for preventing and treating dyskinesia, such as drug-induced dyskinesia in patients of Parkinson's disease. The compositions, preferably in a sustained release formulation, include L-DOPA, carbidopa, and ranitidine wherein the weight ratio of L-DOPA to carbidopa is in the range of about 1:(0.2-0.3), or include L-DOPA, benserazide, and ranitidine, wherein the weight ratio of L-DOPA to benserazide is in the range of about 1:(0.4-0.6).

### CROSS REFERENCE TO RELATED APPLICATIONS

This application claims the benefit under 35 U.S.C. § 119(e) of U.S. Provisional Application Ser. No. 62/548,303, filed Aug. 21, 2017, the content of which is incorporated by reference in its entirety into the present disclosure.

### BACKGROUND

Parkinson's disease (PD) is a devastating neurodegenerative disease caused by the death of dopamine-secreting (“dopaminergic”) neurons in the substantia nigra of the basal ganglia region of the brain. The most widely used treatment for PD is administration of L-DOPA (levodopa, or L-3,4-dihydroxyphenylalanine), the metabolic precursor for dopamine. L-DOPA is converted to dopamine in the brain and various parts of the body by the enzyme DOPA decarboxylase. L-DOPA is used rather than dopamine itself because, unlike dopamine, L-DOPA is capable of crossing the blood-brain barrier. L-DOPA is often co-administered with an enzyme inhibitor of peripheral decarboxylation, such as carbidopa or benserazide, to reduce the amount converted to dopamine in the periphery and thereby increase the amount of L-DOPA that enters the brain.

When L-DOPA is administered regularly over a long period, a variety of unpleasant side effects such as dyskinesia often begin to appear. Dyskinesia refers to a group of movement disorders characterized by involuntary muscle movements. Symptoms of dyskinesia range from slight tremors of the hands to uncontrollable movements of the upper body or lower extremities. Discoordination can also occur internally especially with the respiratory muscles. Few options are available for the pharmacological management of L-DOPA induced dyskinesia (LID), in large part due to the inadequacy of the mechanistic understanding of the syndrome.

### SUMMARY

It is discovered herein that when L-DOPA and carbidopa or benserazide are combined at a specific ratio, presented in a sustained release formulation and used along with ranitidine, the combinatory treatment is useful in preventing and treating drug-induced dyskinesia. The weight ratio of L-DOPA to carbidopa, for example, can be about 1:0.25; and the weight ratio of L-DOPA to benserazide is preferably about 1:0.5. In some embodiments, the L-DOPA can be used at a similar amount as the ranitidine (i.e., weight ratio at about 1:1). The sustained release formulation, in some embodiment, comprises a polymer in which the drugs are dispersed. Non-limiting examples of the polymer include gelatin, a polymer of acrylic acid, a polymer of methyl methacrylate, chitosan, pullulan, and combinations thereof. The drugs can be formulated in a single sustained release formulation or formulated in one or more separate sustained release formulations.

The present disclosure, in one embodiment, provides a formulation, comprising L-DOPA, carbidopa, and ranitidine, wherein the weight ratio of L-DOPA to carbidopa is in the range of about 1:(0.2-0.3). In some embodiments, the L-DOPA, carbidopa, and ranitidine are dispersed in a polymer. In some embodiments, the weight ratio of L-DOPA to carbidopa is in the range of about 1:(0.24-0.26). In some embodiments, the weight ratio of L-DOPA to carbidopa is about 1:0.25.

In another embodiment, the present disclosure provides a sustained release formulation, comprising L-DOPA, benserazide, and ranitidine, wherein the weight ratio of L-DOPA to benserazide is in the range of about 1:(0.4-0.6). In some embodiments, the L-DOPA, benserazide, and ranitidine are dispersed in a polymer. In some embodiments, the weight ratio of L-DOPA to benserazide is in the range of about 1:(0.45-0.55). In some embodiments, the weight ratio of L-DOPA to benserazide is in the range of about 1:0.5.

In some embodiments, the weight ratio of L-DOPA to ranitidine is the range of about 1:(0.75-1.25).

In some embodiments, the polymer is selected from the group of gelatin, a polymer of acrylic acid, a polymer of methyl methacrylate, chitosan, pullulan, and combinations thereof. In some embodiments, the polymer comprises gelatin type A. In some embodiments, the weight ratio of L-DOPA to the polymer is in the range of about 1:(0.75-1.25). In some embodiments, the formulation is in the form of a tablet or a capsule. In some embodiments, the formulation further comprises a pharmaceutically acceptable carrier.

Also provided, in one embodiment, is a formulation, comprising L-DOPA, carbidopa, and ranitidine dispersed in a gelatin, wherein the weight ratio of L-DOPA:carbidopa:ranitidine:gelatin is about 1:0.25:1:1. Also provided, in one embodiment, is a formulation, comprising L-DOPA, benserazide, and ranitidine dispersed in a gelatin, wherein the weight ratio of L-DOPA: benserazide:ranitidine:gelatin is about 1:0.5:1:1.

Methods for preventing or treating dyskinesia in a patient suffering from Parkinson's disease (PD) are also provided. In one embodiment, the method comprises administering to the patient an effective amount of any formulation of the present disclosure. In some embodiments, the patient suffers from dyskinesia induced by a prior treatment of L-DOPA.

Another embodiment provides a method of preventing or treating dyskinesia in a patient suffering from Parkinson's disease (PD), comprising administering to the patient L-DOPA, carbidopa, and ranitidine, wherein the L-DOPA and the carbidopa are dispersed in a polymer and have a weight ratio in the range of about 1:(0.2-0.3). In some embodiments, the L-DOPA and the carbidopa are administered in separate formulations or in a single formulation.

Another embodiment provides a method of preventing or treating dyskinesia in a patient suffering from Parkinson's disease (PD), comprising administering to the patient L-DOPA, benserazide, and ranitidine, wherein the L-DOPA and the benserazide are dispersed in a polymer and have a weight ratio in the range of about 1:(0.4-0.6). In some embodiments, the L-DOPA and the benserazide are administered in separate formulations or in a single formulation.

### BRIEF DESCRIPTION OF THE DRAWINGS

FIG. 1 shows the striatal neuron survival with acute dopamine treatment with and without 3 μM of Ranitidine in accordance with various embodiments.

FIG. 2 shows the results of Abnormal Involuntary Movements (AIMS) study in unilateral 6-hydroxydopamine lesioned (6-OHDA) mice using standard L-DOPA and Gelatin/L-DOPA/Ranitidine formulations in accordance with various embodiments.

FIG. 3 shows the results of Abnormal Involuntary Movements (AIMS) study in unilateral 6-hydroxydopamine lesioned (6-OHDA) mice using standard L-DOPA and Gelatin/L-DOPA/Ranitidine formulations in accordance with various embodiments.

FIG. 4 shows the results of Abnormal Involuntary Movements (AIMS) study in unilateral 6-hydroxydopamine lesioned (6-OHDA) mice using standard L-DOPA and Gelatin/L-DOPA/Ranitidine formulations in accordance with various embodiments.

FIGS. 5A-F show the results of Abnormal Involuntary Movements (AIMs) study after H2 blockade in accordance with various embodiments.

FIG. 6A illustrates an experimental sequence for studying involuntary movement in accordance with various embodiments.

FIG. 6B shows a cylinder test apparatus in accordance with various embodiments.

FIGS. 6C-D show results of contralateral paw usage studies in accordance with various embodiments.

FIG. 6E illustrates locomotor/rotational behavior for scoring in accordance with various embodiments.

FIGS. 7A-E illustrate GRK2, GRK3, GRK5, and GRK6 expression levels observed in hemiparkinsonian mice brain samples in accordance with various embodiments.

FIGS. 7F-J show western blot images of TH, GRK2, GRK3, GRK5, and GRK6 expression levels observed in hemiparkinsonian mice brain samples in accordance with various embodiments.

FIG. 8A shows percentage mice survival in histamine-deficient mice in accordance with various embodiments.

FIGS. 8B-E show the expression profiles of GRK2, GRK3, GRK5, and GRK6 in Hdc-KO mice and their wt littermates in accordance with various embodiments.

FIG. 8F shows percent change in GRK proteins, relative to the intact hemisphere in accordance with various embodiments.

FIG. 8G shows a western blot image of TH expression levels observed in intact and lesioned hemispheres of the Hdc-KO and wt littermates in accordance with various embodiments.

FIG. 8H shows immunostaining for TH in striatum and substantia nigra in accordance with various embodiments.

FIG. 8I shows western blot images of GRK2, GRK3, GRK5, and GRK6 expression levels observed in Hdc-KO mice and their wt littermates in accordance with various embodiments.

FIG. 9A shows the quantitation of the ppERK in the intact and lesioned hemispheres of the experimental groups in accordance with various embodiments.

FIG. 9B shows western blot quantitation of phospho p38 in the intact and lesioned hemispheres of the striatum in accordance with various embodiments.

FIG. 9C shows levels of phospho SAPK/JNKs p46 and p54 in the intact and lesioned hemispheres of the experimental groups in accordance with various embodiments.

FIG. 9D shows phosphorylation and expression data from FIGS. 9A-9C expressed as % expression in the lesioned side relative to the intact side in accordance with various embodiments.

FIG. 9E shows western blots of ppERK and total ERK levels observed in accordance with various embodiments.

FIG. 9F shows western blots of phospho p38 and total p38 levels observed in accordance with various embodiments.

FIG. 9G shows western blots of phospho SAPK/JNK and total SAPK/JNK levels observed in accordance with various embodiments.

FIG. 10A shows a graphical representation of quantitation of phospho Akt at the major (T308) and minor (S473) phosphorylation sites in experimental groups in accordance with various embodiments.

FIG. 10B shows western blot images for pAkt-T308 and pAkt-S473 along with total Akt levels observed in accordance with various embodiments.

FIG. 10C shows the quantitation of Δ Fos B accumulation in the lesioned hemisphere of the brain in experimental groups in accordance with various embodiments.

FIG. 10D shows western blot images for Δ Fos B levels observed in accordance with various embodiments.

FIG. 11A-D show images of ChAT immunoreactivity in the CINs in the intact and lesioned side of DOPA and Ranitidine/DOPA formulation groups in accordance with various embodiments.

FIG. 11E illustrates quantification of fluorescence intensity of ChAT immunoreactivity in accordance with various embodiments.

FIG. 12A shows a magnified image of a lesioned striatum in accordance with various embodiments.

FIGS. 12B-M show immunohistochemical co-localization of pERK and H2R in FoxP1-positive MSNs and CINs in DOPA treated hemiparkinsonian mice in accordance with various embodiments.

FIGS. 12N-12U show immunohistochemical localization of pERK, H2R in ChAT positive interneurons in ranitidine formulation treated hemiparkinsonian mice in accordance with various embodiments.

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Combination Formulations for Prevention and Treatment of Dyskinesia

As demonstrated in the experimental examples, when L-DOPA and a DOPA decarboxylase inhibitor (e.g., carbidopa or benserazide) are combined at a specific ratio, in particular when ranitidine is also used, the combination can effectively prevent, suppress or treat dyskinesia, such as drug-induced dyskinesia in patients suffering from Parkinson's disease (PD).

L-DOPA (L-3,4-dihydroxyphenylalanine) is an amino acid that is the precursor to the neurotransmitters dopamine, norepinephrine (noradrenaline), and epinephrine (adrenaline) collectively known as catecholamines. L-DOPA can be manufactured and in its pure form is sold as a psychoactive drug with the INN levodopa. Trade names include Sinemet, Pharmacopa, Atamet, Stalevo, Madopar, and Prolopa. L-DOPA has a chemical structure of:

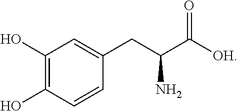 [SMILES: N[C@@H](Cc1ccc(O)c(O)c1)C(=O)O]

Carbidopa is an inhibitor of aromatic amino acid decarboxylation. Carbidopa is a white, crystalline compound, slightly soluble in water, with a molecular weight of 244.3. It is designated chemically as N-amino-α-methyl-3-hydroxy-L-tyrosine monohydrate. The combinations of carbidopa and L-DOPA are marketed under the names Kinson, Sinemet, Pharmacopa and Atamet. Carbidopa has a chemical structure of:

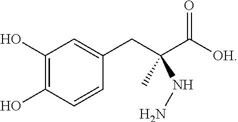 [SMILES: C[C@@](Cc1ccc(O)c(O)c1)(NN)C(=O)O]

Benserazide (also known as Serazide or Ro 4-4602) is a peripherally-acting DOPA decarboxylase inhibitor. Benserazide has a chemical name of (RS)-2-Amino-3-hydroxy-N′-(2,3,4-trihydroxybenzyl)propanehydrazide. It is used in combination with L-DOPA as co-beneldopa (BAN), under the brand names Madopar in the UK and Prolopa in Canada. Benserazide has a chemical structure of:

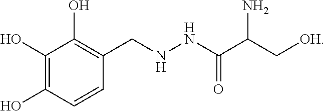 [SMILES: NC(CO)C(=O)NNCc1ccc(O)c(O)c1O]

Ranitidine, sold under the trade name Zantac, can decrease stomach acid production. It is commonly used in treatment of peptic ulcer disease, gastroesophageal reflux disease, and Zollinger-Ellison syndrome. Ranitidine has a chemical name of N-(2-[(5-[(dimethylamino)methyl]furan-2-yl)methylthio]ethyl)-N′-methyl-2-nitroethene-1,1-diamine and a chemical structure of:

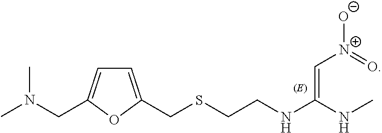 [SMILES: CN/C(=C\[N+](=O)[O-])NCCSCc1ccc(CN(C)C)o1]

In accordance with one embodiment of the present disclosure, a formulation is provided that includes L-DOPA, carbidopa, and ranitidine. The weight ratio of L-DOPA to carbidopa in such a formulation, in some embodiments, is in the range of about 1:(0.2-0.3). In some embodiments, the weight ratio is 1:(0.21-0.29), 1:(0.22-0.28), 1:(0.23-0.27), 1:(0.24-0.26), or 1:(0.245-0.55). In some embodiments, the weight ratio is about 1:0.21, 1:0.22, 1:0.23, 1:0.24, 1:0.25, 1:0.26, 1:0.27, 1:0.28 or 1:0.29. In some embodiments, the weight ratio is about 1:0.25.

In accordance with another embodiment of the present disclosure, a formulation is provided that includes L-DOPA, benserazide, and ranitidine. The weight ratio of L-DOPA to benserazide in such a formulation, in some embodiments, is in the range of about 1:(0.4-0.6). In some embodiments, the weight ratio is 1:(0.41-0.59), 1:(0.42-0.58), 1:(0.43-0.57), 1:(0.44-0.56), 1:(0.45-0.55), 1:(0.46-0.54), 1:(0.47-0.53), 1:(0.48-0.52), or 1:(0.49-0.51). In some embodiments, the weight ratio is about 1:0.45, 1:0.46, 1:0.47, 1:0.48, 1:0.49, 1:0.5, 1:0.51, 1:0.52, 1:0.53, 1:0.54, or 1:0.55. In some embodiments, the weight ratio is about 1:0.5.

As shown in the experimental examples, ranitidine can reduce the toxicity of 6-OHDA has on neurons and, when used together with L-DOPA and a DOPA decarboxylase inhibitor, can reduce dyskinesia. In one embodiment, the weight ratio of L-DOPA to ranitidine in any of the above formulations is in the range of about 1:(0.75-1.25). In some embodiments, the weight ratio of L-DOPA to ranitidine is in the range of about 1:(0.8-1.2), 1:(0.86-1.1.5), 1:(0.9-1.1), 1:(0.95-1.05), 1:(0.98-1.02), or 1:(0.99-1.01). In some embodiments, the weight ratio of L-DOPA to ranitidine is about 1:0.85, 1:0.9, 1:0.95, 1:0.98, 1:0.99, 1:1, 1:1.01, 1:1.02, 1:1.05, 1:1.1, or 1:1.15. In some embodiments, the weight ratio of L-DOPA to ranitidine is about 1:1.

The formulations, in some embodiments, are prepared in sustained release forms. Various types of sustained release forms are further described in the specification below, some of which use pharmaceutical suitable polymers. Non-limiting examples of such polymers include gelatin (e.g., type A), a polymer of acrylic acid, a polymer of methyl methacrylate, chitosan, pullulan, and combinations thereof. In some embodiments, the sustained release forms can provide controlled release of the contained drug or drugs over an extended period of time, which is at least 30 minutes, 1 hour, 2 hours, 4 hours, 8 hours, 12 hours, 24 hours, 36 hours, 48 hours, 3 days, 5 days, 7 days, 2 weeks, or 4 weeks.

Gelatin is a mixture of peptides and proteins produced by partial hydrolysis of collagen extracted from the skin, bones, and connective tissues of animals such as domesticated cattle, chicken, pigs, and fish. During hydrolysis, the natural molecular bonds between individual collagen strands are broken down into a form that rearranges more easily. Its chemical composition is, in many aspects, closely similar to that of its parent collagen. Photographic and pharmaceutical grades of gelatin generally are sourced from cattle bones and pig skin. Commercial gelatin obtained from acid-treated raw material has been called type-A gelatin and the gelatin obtained from alkali-treated raw material is referred to as type-B gelatin.

When gelatin is used, in one embodiment, the weight ratio of L-DOPA to gelatin in any of the above formulations is in the range of about 1:(0.75-1.25). In some embodiments, the weight ratio of L-DOPA to gelatin is in the range of about 1:(0.8-1.2), 1:(0.86-1.1.5), 1:(0.9-1.1), 1:(0.95-1.05), 1:(0.98-1.02), or 1:(0.99-1.01). In some embodiments, the weight ratio of L-DOPA to gelatin is about 1:0.85, 1:0.9, 1:0.95, 1:0.98, 1:0.99, 1:1, 1:1.01, 1:1.02, 1:1.05, 1:1.1, or 1:1.15. In some embodiments, the weight ratio of L-DOPA to gelatin is about 1:1.

In a particular embodiment, a formulation is provided that includes L-DOPA, carbidopa, and ranitidine dispersed in a gelatin, wherein the weight ratio of L-DOPA:carbidopa:ranitidine:gelatin is about 1:0.25:1:1. In another embodiment, a formulation is provided that includes L-DOPA, benserazide, and ranitidine dispersed in a gelatin, wherein the weight ratio of L-DOPA: benserazide:ranitidine:gelatin is about 1:0.5:1:1.

Methods and manufacturing uses of the formulations are also provided. As demonstrated in the examples, the formulations of the present disclosure can reduce the symptoms of dyskinesia. Dyskinesia refers to a group of movement disorders characterized by involuntary muscle movements. Symptoms of dyskinesia range from slight tremors of the hands to uncontrollable movements of the upper body or lower extremities. Discoordination can also occur internally especially with the respiratory muscles.

Therefore, in one embodiment, provided is a method for preventing, inhibiting or treating dyskinesia. In some embodiments, the method entails administering to a patient in need thereof a formulation of any embodiment of the present disclosure. In some embodiment, the patient suffers from Parkinson's disease (PD). In some embodiments, the patient has been treated with a therapy targeting the PD. In some embodiments, the patient has received a treatment with L-DOPA, optionally with a DOPA decarboxylase inhibitor.

The different active ingredients (e.g., L-DOPA, carbidopa/benserazide, and ranitidine) can also be administered separately. For instance, each of the L-DOPA, carbidopa/benserazide, and ranitidine can be formulated in a separate dosage form. In another example, the L-DOPA and carbidopa or benserazide can be formulated together, while ranitidine is formulated separately. In another example, the L-DOPA and ranitidine are formulated together and the carbidopa or benserazide is formulated separately. In some embodiments, the ranitidine and carbidopa or benserazide can be formulated together, while L-DOPA is formulated separately. The formulation can include the polymer as described above.

Whether these ingredients are formulated together or separately, in one embodiment, the present disclosure provides a method of preventing or treating dyskinesia in a patient in need thereof, which entails administering to the patient L-DOPA, carbidopa, and ranitidine, wherein the L-DOPA and the carbidopa are dispersed in a polymer and have a weight ratio in the range of about 1:(0.2-0.3). In another embodiment, the present disclosure provides a method of preventing or treating dyskinesia in a patient in need thereof, which entails administering to the patient L-DOPA, benserazide, and ranitidine, wherein the L-DOPA and the benserazide are dispersed in a polymer and have a weight ratio in the range of about 1:(0.4-0.6).

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Formulations may, in some embodiments, be administered to a subject (including a human) who is at risk or has a family history of the disease or condition.

“Patient” or “subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of dyskinesia. The therapeutically effective amount may vary depending on the subject, and disease or condition being treated, the weight and age of the subject, the severity of the disease or condition, and the manner of administering, which can readily be determined by one or ordinary skill in the art.

### Sustained Release Forms

In the above description, gelatin is used as an example for providing a sustained-release dosage form. A sustained release form can provide controlled release of the contained drug or drugs over an extended period of time (e.g., over 4 hours, 8 hours, 12 hours, 24 hours, etc.). A sustained-release dosage form comprises a release rate-controlling material. The rate-controlling material can be associated with the formulation either in the form of a matrix or a coating. The rate-controlling material is a material that permits release of the active agent at a sustained rate into an aqueous medium. In some embodiments, the rate-controlling component is hydrophilic, hydrophobic, enteric, or combinations thereof.

Suitable hydrophilic materials comprise water soluble or water swellable materials. Examples of such materials include hydroxyalkyl celluloses, hydroxyalkyl alkylcelluloses, and carboxyalkyl cellulose esters, for example, hydroxypropyl methylcelluloses (hypromelloses or HPMC), hydroxypropylcelluloses (HPC), and combinations comprising one or more of the foregoing materials. For the purposes of this disclosure, the release modifying agent may be present in a matrix, or in a coating covering the matrix.

In some embodiments, pharmaceutical compositions comprise mixtures of water soluble materials of different viscosity grades, such as hydroxypropyl methylcelluloses and hydroxypropylcelluloses. These water soluble materials may be characterized by their viscosities in a 2% w/w aqueous solution as low viscosity (less than about 1 Pa·s, or less than about 1,000 cP), medium viscosity (about 1 Pa·s to about 10 Pa·s, or about 1,000 cP to about 10,000 cP), and high viscosity (greater than about 10 Pa·s, or greater than about 10,000 cP).

Hydroxypropyl methylcellulose polymers that are hydrophilic in nature and may be used in the present disclosure are sold in different viscosity grades such as those sold under the brand name Methocel™ available from Dow Chemical Co. Examples of hydroxypropyl methylcellulose polymers of a low viscosity grade include those available under the brand names Methocel E5, Methocel E-15 LV, Methocel E50 LV, Methocel K100 LV and Methocel F50 LV whose 2% by weight aqueous solutions have viscosities of 5 cP, 15 cP, 50 cP, 100 cP, and 50 cP, respectively. Examples of hydroxypropyl methylcellulose polymers having medium viscosity include those available under the brand names Methocel E4M and Methocel K4M, both of whose 2% by weight aqueous solutions have a viscosity of 4000 cP. Examples of hydroxypropyl methylcellulose polymers having high viscosity include those available under the brand names Methocel K15M and Methocel K100M whose 2% by weight aqueous solutions have viscosities of 15,000 cP and 100,000 cP, respectively. The hydroxypropyl methylcellulose polymers may be present in the pharmaceutical compositions of the present disclosure in amounts from about 0.1% to 50% by weight.

The hydroxypropylcellulose polymers that may be used in the present disclosure also include, for example, polymers available under the brand name Klucel™, available from Nippon Soda Co. Hydroxypropylcellulose polymers available under the brand names Klucel EF, Klucel LF, Klucel JF and Klucel GF, whose 2% by weight aqueous solutions have viscosities less than 1000 cP, are examples of low viscosity hydrophilic polymers. A hydroxypropylcellulose polymer available under the brand name Klucel ME whose 2% by weight aqueous solution has a viscosity in the range from 4,000-6,500 cP is a medium viscosity hydrophilic polymer. Hydroxypropyl cellulose polymers available sold as HPC-SL, HPC-L, and HPC-M, whose 2% by weight aqueous solutions have viscosities of 3-6 cP, 6-10 cP, and 150-400 cP, respectively, are examples of low viscosity hydrophilic polymers, while HPC-H has a viscosity of 1,000-4000 cP and is an example of a medium viscosity hydrophilic polymer. The hydroxypropylcellulose polymers may be present in an amount from about 0.1% to 50% by weight.

Water swellable substances suitable for making sustained release dosage forms are compounds that are able to expand when they are exposed to aqueous fluids, such as gastro-intestinal fluids. One or more water swellable substances may be present in a matrix or coating together with the active agent and optionally one or more pharmaceutically acceptable excipients.

Suitable substances which can be used as water swellable substances include, for example, low-substituted hydroxypropyl celluloses, e.g. L-HPC, cross-linked polyvinylpyrrolidones, e.g., PVP-XL, Kollidone™ CL and Polyplasdone™ XL, sodium carboxymethylcellulose, cross-linked sodium carboxymethylcellulose, e.g., Ac-di-Sol™ and Primenose™, sodium starch glycolate, e.g., Primojel™ sodium carboxymethylcelluloses, e.g., Nymcel™ ZSB10, sodium carboxymethyl starches, e.g., Explotab™, ion-exchange resins, e.g., Dowex™ or Amberlite™ products, microcrystalline cellulose, e.g., Avicel™ products, starches and pregelatinized starches, e.g., Starch 1500™ and Sepistab ST200™, formalin-casein, e.g., Plas-Vita™, and combinations comprising one or more of the foregoing water swellable substances.

In embodiments, hydrophilic materials include polyalkylene oxides, polysaccharide gums, and crosslinked polyacrylic acids.

Suitable polyalkylene oxides, such as linear polymers of unsubstituted ethylene oxide, include Polyox™ products from The Dow Chemical Company, U.S., having molecular weights about 100,000-7,000,000. For example, poly(ethylene oxide)polymers having molecular weights about 4,000,000 and higher, such as about 4,500,000 to about 10,000,000, or about 5,000,000 to about 8,000,000, can be used. Other useful polyalkylene oxide polymers are made from propylene oxide, or mixtures of ethylene oxide and propylene oxide.

Polysaccharide gums, both natural and modified (semi-synthetic), can be used. Examples are dextran, xanthan gum, gellan gum, welan gum and rhamsan gum.

Crosslinked polyacrylic acids that can be used include those having properties similar to those described above for alkyl-substituted cellulose and polyalkylene oxide polymers. Useful crosslinked polyacrylic acids include those with viscosities about 4,000 to about 40,000 cP (for a 1% aqueous solution at 25° C.). Three specific examples are CARBOPOL™ grades 971P, 974P, and 934P (sold by The Lubrizol Corporation, Cleveland, Ohio, USA). Further examples are polymers known as WATER LOCK™, which are starch/acrylate/acrylamide copolymers available from Grain Processing Corporation, Muscatine, Iowa, USA.

The hydrophilicity and water swellability of these polymers cause the active agent-containing matrices to swell in size after oral administration, due to ingress of water. The release rate of an active agent from the matrix is primarily dependent upon the rate of water imbibition and the rate at which the active agent dissolves and diffuses from the swollen polymer, which in turn is related to the solubility and dissolution rate of the active agent, the active agent particle size, and the active agent concentration in the matrix.

Suitable “hydrophobic” materials are water-insoluble neutral or synthetic waxes, fatty alcohols such as lauryl, myristyl, stearyl, cetyl, or cetostearyl alcohol, fatty acids and derivatives thereof, including fatty acid esters such as such as glyceryl monostearate, glycerol monooleate, acetylated monoglycerides, stearin, palmitin, laurin, myristin, cetyl esters wax, glyceryl palmitostearate, glyceryl behenate, hydrogenated castor oils, cottonseed oils, fatty acid glycerides (mono-, di-, and tri-glycerides), hydrogenated fats, hydrocarbons, normal waxes, stearic acid, stearyl alcohol, materials having hydrocarbon backbones, and combinations comprising one or more of the foregoing materials. Suitable waxes include, but are not limited to, beeswax, Glycowax® (a N,N′-distearoylethyelenediamine, from Lonza), castor wax, carnauba wax, and wax-like substances.

A wax formulation is a solid dosage form comprising the in a waxy matrix. The waxy matrix may be prepared by known tableting technologies such as wet granulation, dry granulation, or direct compression. Alternatively, a waxy matrix may be prepared by melting a suitable wax material and using the melt to granulate the active agent, optionally in combination with one or more other excipient materials. The matrix material comprises the waxy material and the active agent.

The wax material can be, for example, an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those having skill in the art. A typical cetyl esters wax, for example, has a molecular weight of about 470 to about 490 and is a mixture containing primarily esters of saturated fatty alcohols and saturated fatty acids.

The wax material can comprise a vegetable wax such as carnauba wax, a hydrogenated castor oil, glyceryl behenates, and combinations comprising one or more of the foregoing waxes. Hydrogenated castor oil is a hard wax with a high melting point, about 83-88° C. Hydrogenated castor oil is obtained by hydrogenation of virgin castor oil. It is mainly the triglyceride of 12-hydroxystearic acid.

When the waxy material is a hydrogenated castor oil and no other waxy material is used, the matrix can be coated with a functional coating. When the waxy material includes glyceryl behenates or carnauba wax, the matrix can be used without a coating, but may have either a cosmetic coating or a functional coating depending on the precise release profile and appearance desired. Sometimes combinations of waxes such as carnauba wax and glyceryl behenate, carnauba wax and castor wax, etc., may be used.

In some embodiments, the formulations include a rate-controlling material that is an “enteric polymer,” being insoluble in highly acidic environments such as the stomach, but being dissolved or decomposed in higher pH environments such as the intestines. Examples include polyvinylacetate phthalates (PVAP), alginic acid and its derivatives, hydroxypropyl methylcellulose acetate succinates (HPMCAS), cellulose acetate phthalates (CAP), methacrylic acid copolymers, hydroxypropyl methylcellulose succinates, cellulose acetate succinates, cellulose acetate hexahydrophthalates, hydroxypropyl methylcellulose hexahydrophthalates, hydroxypropyl methylcellulose phthalates (HPMCP), cellulose propionate phthalates, cellulose acetate maleates, cellulose acetate trimellitates, cellulose acetate butyrates, cellulose acetate propionates, methacrylic acid/methacrylate polymers (e.g., acid number 300 to 330 and also known as EUDRAGIT™ L from Evonik Industries, Germany, which is an anionic copolymer based on methacrylate, available as a powder, and also known as methacrylic acid copolymer, type A NF), methacrylic acid-methyl methacrylate copolymers, ethyl methacrylate-methylmethacrylate-chlorotrimethylammonium ethyl methacrylate copolymers, and the like, and combinations comprising one or more of the foregoing enteric polymers. Other examples include natural resins, such as shellac, sandarac resin, copal collophorium, and combinations comprising one or more of the foregoing polymers. Further examples of enteric polymers include synthetic resins bearing carboxyl groups. The methacrylic acid-acrylic acid ethyl ester 1:1 copolymer solid substance of the acrylic dispersion sold as EUDRAGIT L-100-55 is suitable.

Polymethacrylate enteric polymers are synthetic cationic and anionic polymers of dimethylaminoethyl methacrylates, methacrylic acid and methacrylic acid esters in varying molar ratios. Several different types are commercially available and may be purchased as dry powders, or in aqueous mixtures.

### Kits

Provided herein are also kits that include one or more ingredients (e.g., L-DOPA, carbidopa, benserazide, and/or ranitidine) of the disclosure, and suitable packaging. In one embodiment, a kit further includes instructions for use. In one aspect, a kit includes one or more ingredients of the disclosure, and a label and/or instructions for use of the ingredients in the treatment of the indications, including the diseases or conditions, described herein.

Provided herein are also articles of manufacture that include one or more ingredients described herein in a suitable container. The container may be a vial, jar, ampoule, preloaded syringe, and intravenous bag.

### Pharmaceutical Dosage Forms and Modes of Administration

Provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compound (e.g., L-DOPA) of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

### Examples

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Abbreviations Used in the Examples

6-OHDA, 6-hydroxydopamine, AIMs—Abnormal Involuntary Movements, ALO AIMs—Axial Limb Orofacial Abnormal Involuntary Movements, Akt—Protein Kinase B, ANOVA—Analysis of Variance, APLAC—Administrative Panel on Laboratory Animal Care, BSA—Bovine Serum Albumin, ChAT—Choline Acetyl Transferase, CINs—Cholinergic Interneurons, DOPA—L-3,4-dihydroxyphenylalanine, DA—Dopamine, D1R—Dopamine D1 Receptor, D2R—Dopamine D2 Receptor, ECL—Electrochemical luminescence, ERK—Extracellular Signal Regulated Kinase, FI—Fluorescence intensity, LID—L-DOPA Induced Dyskinesia, GRK—G protein coupled receptor kinase, GRK2—G protein coupled receptor kinase2, GRK3—G protein coupled receptor kinase3, GRK5—G protein coupled receptor kinase5, GRK6—G protein coupled receptor kinase6, GPCR—G protein coupled receptor, HA—Histamine, Hdc-KO-Histidine decarboxylase knockout, H1R—Histamine H1 receptor, H2R—Histamine H2 receptor, H3R—Histamine H3 receptor, H4R—Histamine H4 receptor, H+L—Heavy+Light chain of Immunoglobulin G, JNK—Jun Amino Terminal Kinases, MAPK—Mitogen activated protein kinase₅, MSN—Medium Spiny Neurons, p38—p38 Mitogen Activated Protein Kinase, phospho-p38—Phosphorylated p38 Mitogen Activated Protein Kinase, PBS—Phosphate Buffered Saline, PD—Parkinson's disease, pERK—Phosphorylated Extracellular Signal Regulated Kinase, PVDF—Polyvinylidinefluoride, RGS—Regulator of G Protein Signaling, RM-ANOVA—Repeated Measured Analysis of Variance, SAPK/JNK—Stress Activated Protein Kinases/Jun Amino Terminal Kinases, SEM—Standard Error of Mean, Ser473—Serine 473 site of Akt/Protein kinase B, TBS—Tris Buffered Saline, TBS-T—Tris Buffered Saline—Tween20, Thr308—Threonine 308 site of Akt/Protein kinase B, TH—Tyrosine Hydroxylase, WT—Wild Type.

### Example 1: Ranitidine Reduced L-DOPA-Induced Dyskinesia

Background:

This example shows that ranitidine, at 3 μM, protected neurons against high-dose of dopamine (DA) toxicity in in vitro striatal neuronal culture.

Methods:

Striatal neurons were cultured from rat embryonic E18 pups. Briefly the brains were dissected from the E18 pups and placed in Neurobasal medium. The individual striata were pooled together and digested in papain to dissociate the cells. The dissociated striatal neurons were cultured in Neurobasal medium supplemented with B27 and antibiotics and cultured at 5% CO₂ humidified incubator at 37° C. After 3 weeks, the mature striatal neurons were treated with increasing dose of dopamine with or without the addition of 3 μM of Ranitidine for 24 hrs. As a control, the cultures were treated with 3 μM of Ranitidine alone. The images of the surviving neurons were taken with a bright field phase contrast microscope and the number of surviving and dead cells were counted manually. The rounded cells were considered as apoptotic and dead cells whereas the neurons with prominent axonal and dendritic projections were considered viable and mature cells.

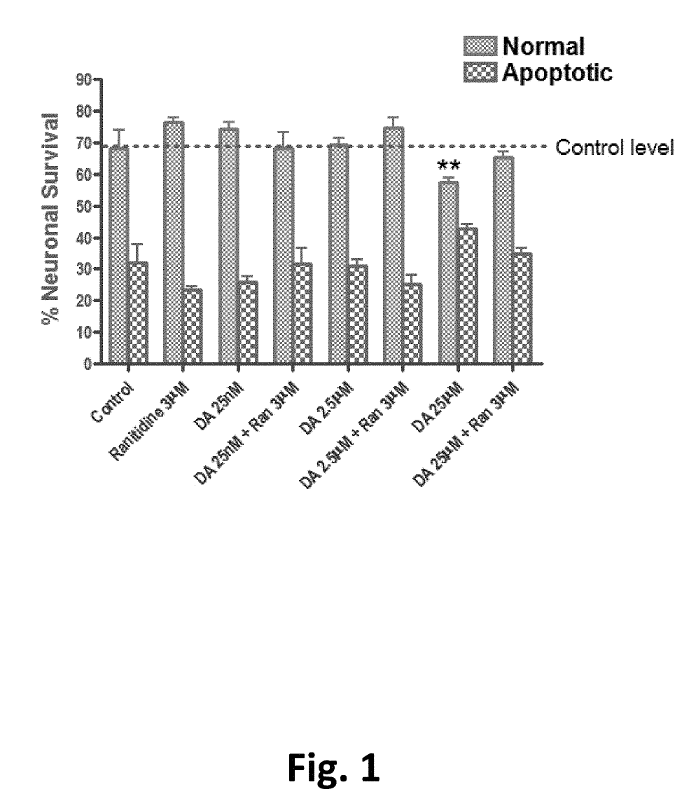

FIG. 1 shows the striatal neuron survival with acute dopamine treatment with and without 3 μM of Ranitidine. The % neuronal survival is plotted against the various treatment groups. In the control group, the cells were not treated and served as the sham group. In the other wells the striatal neurons were challenged with increasing dose of dopamine from 25 nM to 25 μM with and without 3 μM of Ranitidine. In the 25 μM dopamine group there was significant neuronal loss compared to the other treatment groups.

Conclusion:

Ranitidine substitution significantly reduced the neuronal death with higher dose of dopamine. The results suggest that Ranitidine when presented along with higher doses of dopamine serves as a neuroprotective agent against dopamine induced toxicity.

### Example 2: Testing of Combination Formulations in Mice with Drug-Induced Dyskinesia

Background:

This example shows that sustained release formulations of L-DOPA, ranitidine, and a DOPA decarboxylase inhibitor (e.g., carbidopa and benserazide), while at optimal ratios, can effectively suppress drug-induced dyskinesia.

Methods:

Unilateral 6-hydroxydopamine (6-OHDA) lesioned mice were used for studying the Abnormal Involuntary Movements (AIMs) or dyskinetic behavior. The mice were received daily injections of standard L-DOPA (5 mg/Kg) and were scored on 1^{st}, 4^{th} and 7^{th} days of the pretesting session with an interval of 3 days between the testing sessions and were separated into five groups randomly based on the dyskinetic scores.

In the post-testing sessions, the mice were separated into 5 groups and were injected daily with L-DOPA and the respective formulations as shown below and scored every third day for dyskinetic behavior for 5 sessions. Four groups received Gelatin formulations of L-DOPA/Ranitidine with either Carbidopa or Benserazide as the dopamine decarboxylase (DDC) inhibitor. The 5^{th} group served as the control and received standard L-DOPA/Benserazide at a dose of 5 mg DOPA and 10 mg/Kg of Benserazide respectively.

| | | Weight |
|---|---|---|
| Group | Ingredients | Ratio |
| DOPA (control) | L-DOPA, Benserazide | 1:2 |
| Formulation I | Gelatin*, L-DOPA, Carbidopa, Ranitidine | 1:1:0.25:1 |
| Formulation II | Gelatin, L-DOPA, Carbidopa, Ranitidine | 1:1:0.5:1 |
| Formulation III | Gelatin, L-DOPA, Benserazide, Ranitidine | 1:1:0.5:1 |
| Formulation IV | Gelatin, L-DOPA, Benserazide, Ranitidine | 1:1:1:1 |

*Gelatin used here was gelatin type A.

Testing of the dyskinetic Behavior used an Abnormal Involuntary Movement Scale (AIMS) system, which measured oro-lingual, forelimb, axial dystonia, and rotational behavior. During pretesting, the AIMS scoring lasted for a total period of 3 hrs with the scoring done at 20 minutes intervals. The animals received scores from 0 to 4 based on the duration of the dyskinetic behavior and represented as cumulative or total score/animal/session. The scores can be any one of 0-4:

0: No movement
1: <30 secs
2: >30 secs-1 min
3: After 1 min respond to stimulus and stop
4: After 1 min doesn't respond to stimulus.

Kruskal-Wallis non-parametric analysis was used to compare between the pre-testing groups to check for statistical significance among the different DOPA groups before grouping. The Mann-Whitney non-parametric test was used to study the difference between individual groups in the post-testing session and the p value less than 0.05 was considered statistically significant.

Results:

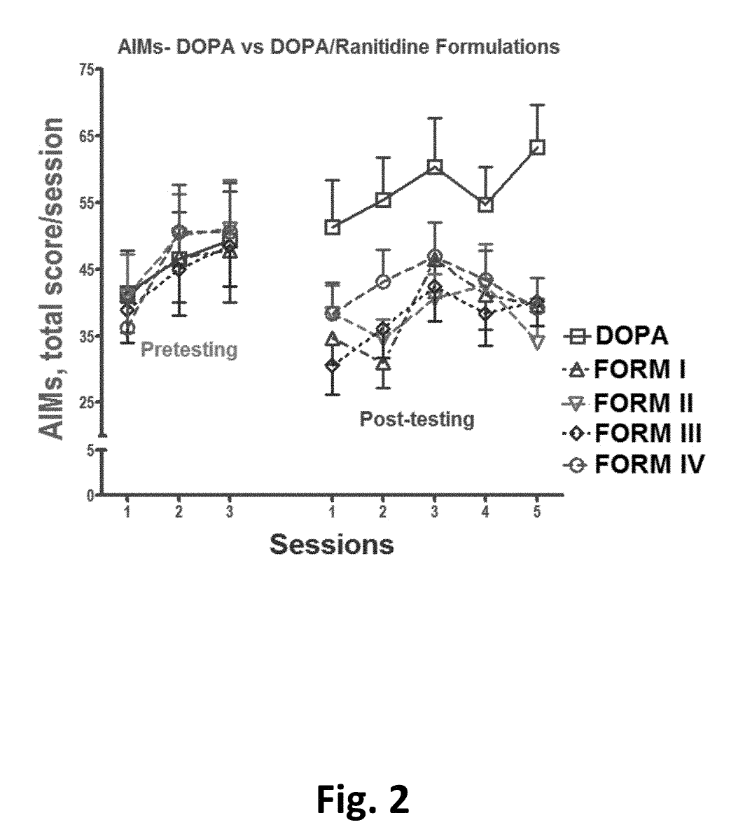

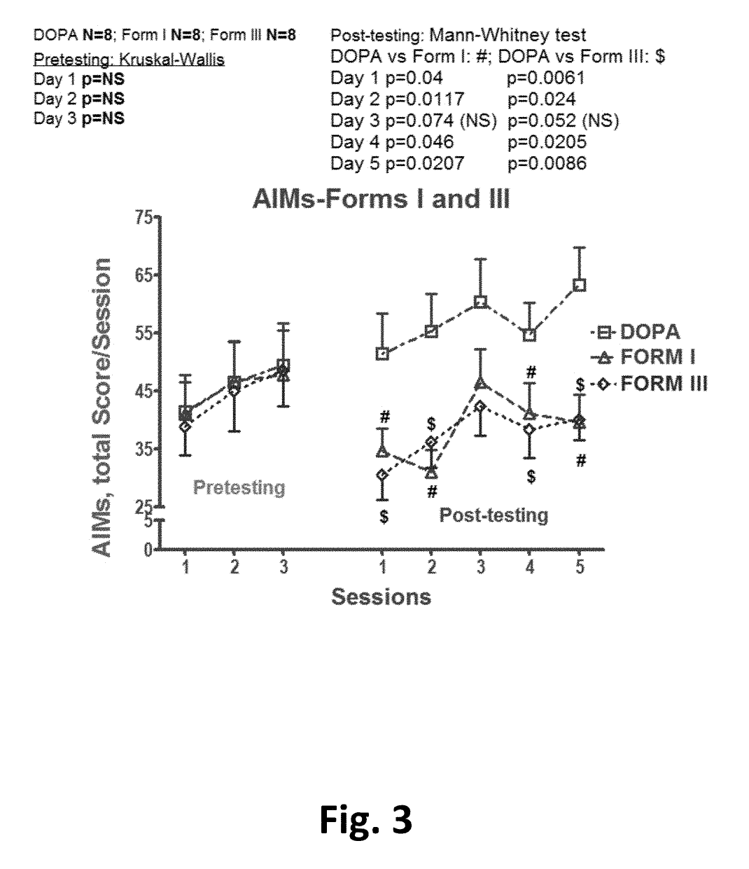

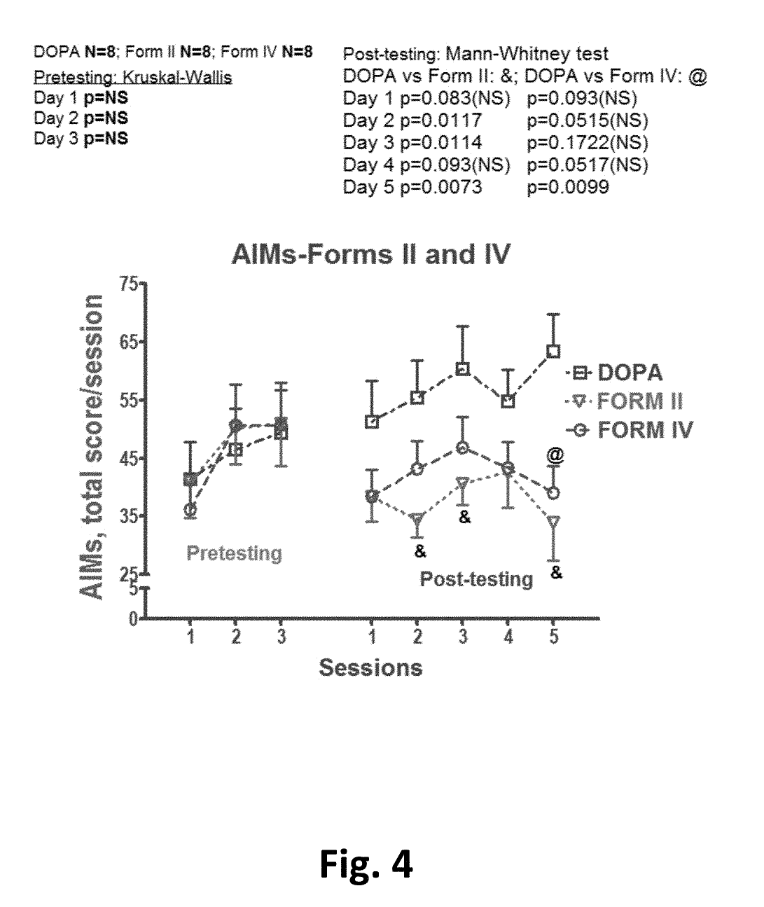

There was no statistical difference observed between the groups in the pre-testing session for the AIMs. However, in the post-testing session the AIMs scores were significantly reduced in the formulation groups I and III on testing sessions 1, 2, 3, and 5 than the control L-DOPA treated groups (FIGS. 2 and 3). In the case of formulation II, significantly reduced AIMs scores were observed on sessions 2, 3, and 5 (FIGS. 2 and 4) than the control DOPA groups whereas significantly reduced AIMs were only observed on session 5 for formulation IV group than the control group. There was no statistical difference among with the different formulation groups compared on the various testing sessions.

Conclusion:

Among the formulations tested, formulations I, II and III had better outcome compared with the standard L-DOPA as per the AIMs study.

### Example 3: Pharmaceutical Upregulation of GRK3 in Dopamine Depleted Striatum Ameliorated L-DOPA Induced Dyskinesia in Parkinson's Disease

Methods: Chronic L-DOPA administration was used to generate LID in a hemiparkinsonian mouse model for testing of ranitidine, a H2R antagonist. Histamine (HA)-deficient mice (Hdc-KO) were used to study the role of histamine in the development of LID. Loss of histamine (HA) in Hdc-KO mice didn't result in the down regulation of G protein coupled receptor kinases (GRKs), especially GRK3 and GRK6 which are found to the significantly reduced in PD animal models when lesioned with 6-OHDA for dopamine (DA) depletion. Ranitidine, when given along with L-DOPA using a gelatinized formulation enhanced the expression of GRK3 in the DA depleted striatum thereby inhibiting LID in mice. The MAPK and Δ Fos B signaling pathways were not attenuated when ranitidine was combined with L-DOPA in the formulations similar to the control DOPA groups. The results in Examples 3-9 demonstrate that ranitidine inhibits LID by normalizing the levels of GRK3 in the DA depleted striatum via HA H2R.

### Unilateral 6-Hydroxydopamine (6-OHDA) Lesioning (the Hemiparkinsonian Mouse Model)

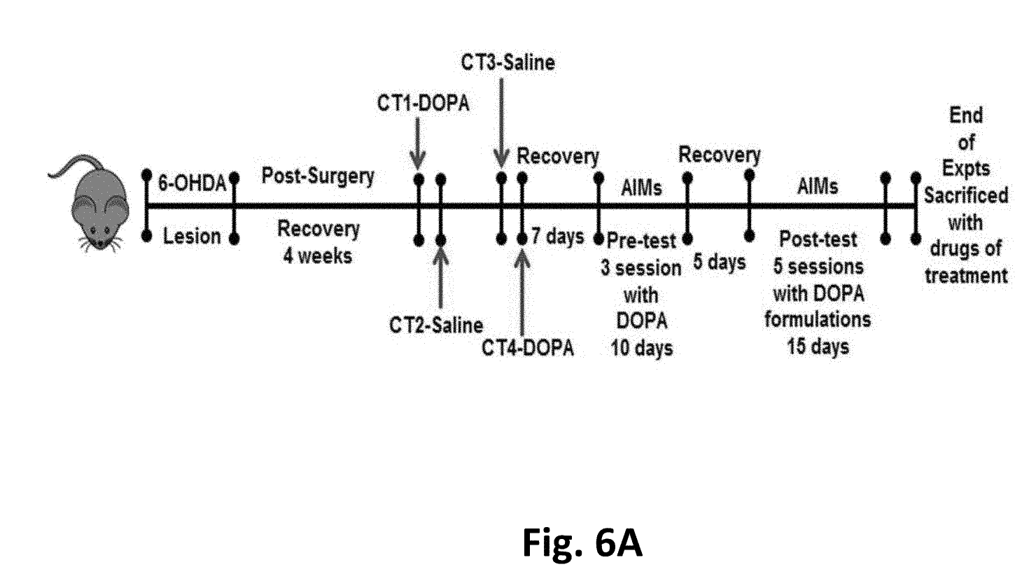

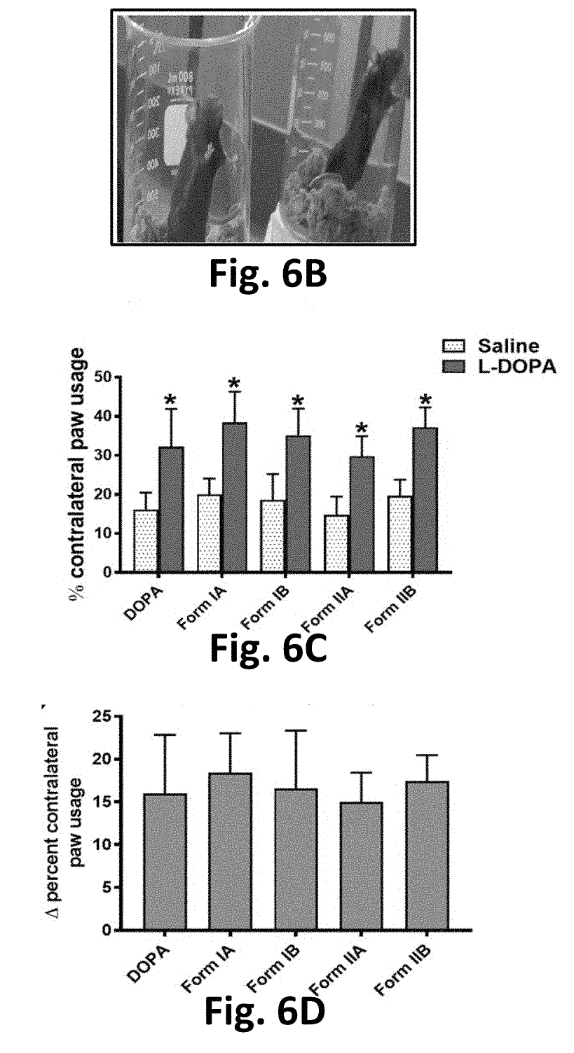

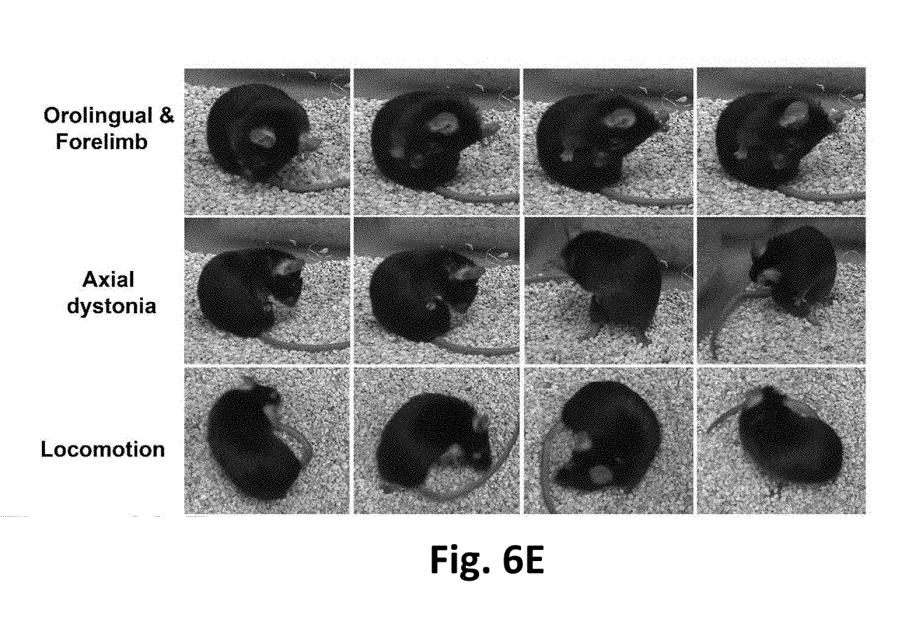

FIG. 6A shows the sequence of manipulations and testing. C57Bl/6 mice from Jackson laboratories, age 12 wks, were used for all studies, with the exception of Hdc-KO studies, for which mice backcrossed extensively onto the C57Bl/6 background were bred in-house; wild-type siblings were used as controls. Adult males were housed in a Stanford University animal facility with 12/12 hr light/dark cycle and had free access to water and food. All procedures followed National Institute of Health guidelines and were approved by the Institutional Administrative Panel on Laboratory Animal Care (APLAC).

Mice were anesthetized with 5% isoflurane (Isothesia, Henry Schein Animal Health, Dublin, Ohio) in 100% oxygen with a delivery rate of 51/minute until loss of righting reflex and mounted on a stereotaxis. The anesthesia was maintained with 1 to 1.5% isoflurane throughout the surgical procedure. Body temperature was maintained using heating pads; respiration was monitored every 10 minutes. Mice were treated with desipramine HCl (25 mg/kg i/p) 30 minutes before the infusion of 6-hydroxydopamine (6-OHDA). 2 μL (2 μg/μL) of 6-OHDA (Santacruz Biotechnology, Santa Cruz, Calif.) was unilaterally infused at the rate of 0.5 μL/min in to the median forebrain bundle (MFBB) at co-ordinates AP=1.1, ML=−1.3, DV=5.02 from the bregma to target dopaminergic projection neurons. Animals were allowed to recover from anesthesia. 4 weeks after lesion surgery, animals were tested for rotational behavior with apomorphine (0.1 mg/Kg). Only animals that exhibited ≥90 contralateral rotations during 1 hr after apomorphine were used in subsequent studies.

Gelatinized oral formulations of L-DOPA, ranitidine, and peripheral DOPA decarboxylase inhibitors (carbidopa and benserazide) were formulated according to a standard protocol using a Buchi spray dryer. The formulations used were as follows:

Ranitidine (500 mg)+Carbidopa (125 mg)+DOPA (500 mg), Form IA:

Ranitidine (500 mg)+Carbidopa (250 mg)+DOPA (500 mg), Form IB:

Ranitidine (500 mg)+Benserazide (250 mg)+DOPA (500 mg), and Form IIA:

Ranitidine (500 mg)+Benserazide (500 mg)+DOPA (500 mg). Form IIB:

The ranitidine/DOPA/DOPA decarboxylase formulations were made and split equally in to two halves and to one set extra DOPA decarboxylase was added to serve as the groups with twice the normal levels of DOPA decarboxylases. All the formulations were then coated with 500 mg of gelatin before the spray drying and the weights of the formulations were normalized equivalent to the dose of DOPA.

### Behavioral Studies

Cylinder test to test paw asymmetry. The cylinder test was used to study the L-DOPA mediated amelioration of akinesia in 6-OHDA lesioned mice (Brooks and Dunnett, 2009). Mice were habituated to the testing room for 30 minutes prior to the experiment. They were then treated with a single dose of saline, L-DOPA/Benserazide combination (5 and 10 mg/Kg respectively by s/c), or the DOPA formulations described above (dose equivalent to 5 mg/Kg of DOPA was used) and immediately placed in a 500 mL clean Pyrex glass beaker, partially filled with bedding material. Video recording was started 2 minutes after placing the mice in the beaker and continued for 10 mins. Touches of the cylinder wall with all digits clearly extended were quantified for the affected (contralateral to the 6-OHDA lesion) and control side. The experimenter and the analyzer of the video tapes were blind to lesion and treatment assignment. Animals were tested twice, once with L-DOPA or a mixed formulation and once with saline, in counterbalanced order, with 1 week between.

Measurement of Abnormal Involuntary Movements (AIMs) were quantified after chronic L-DOPA administration in hemiparkinsonian mice. In the pretesting sessions, mice were given daily injections of L-DOPA/benserazide (5/10 mg/Kg s/c) for 9 days; AIMs were assessed every 20 minutes for 3 hrs after injection every third day of L-DOPA injections. Total duration of contralateral abnormal movements of the tongue-orofacial muscles and forelimb, twisting of the body (dystonia), and locomotor (rotational) behavior were scored, as illustrated in FIG. 6E. After pretesting, mice were separated in to 5 groups with equal AIMs scores. After five days' rest, in the post-test phase, mice were administered DOPA-benserazide or one of the four ranitidine formulations (Form IA, IB, IIA, or IIB, as defined above) daily for 15 days; AIMs were again assessed every third day.

### Sample Preparation and Western Blotting

After behavioral testing, mice again received their assigned injection and then, 45 minutes later, were decapitated under anesthesia. Tissue samples were processed as follows. Briefly, the brain was dissected, rapidly frozen on dry ice, and stored at −80° C. Striatal tissue was collected from 100 μm coronal slices cut at the level of striatum using a cryostat (Leica CM1950, Leica Biosystems Inc, Buffalo Groove, Ill.). Striatal samples from the intact and the lesioned hemisphere were separately collected in 150p1 of lysis solution (Totally RNA, Ambion, Austin, Tex.). Care was taken to make sure that the tissue samples were collected from similar regions in all the brain samples analyzed, since there are differences in the expression of GRKs from rostral to caudal CPu. Protein concentration was estimated using Bradford reagent (Bio-Rad, Hercules, Calif.). 200m protein was precipitated with 100% methanol and centrifuged at 10000 g for 10 mins in a table top centrifuge. The pellet was re-suspended in 90% methanol and centrifuged for an additional 10 mins at 10000 g. The supernatant was discarded and the pellet was air dried and dissolved in 400 μL of β-mercaptoethanol containing 2× Laemmli sample buffer (Biorad, Hercules, Calif.), for a final concentration of 0.5 mg/mL. These samples were stored at −80° C. until use. Samples were electrophoresed through 10% polyacrylamide gels and transferred on to Immobilon-P PVDF membranes (Millipore, Bedford, Mass.) and processed. Sample loading was counterbalanced across experimental groups.

PVDF membranes were blocked with 5% skimmed milk powder for 1 hr at room temp and probed with the primary antibodies. Blots were washed with TBS-T to remove milk and incubated with primary antibodies overnight at 4° C. and then with horse radish peroxidase-conjugated goat anti-rabbit or rabbit anti-mouse (H+L) secondary antibodies (Jackson ImmunoResearch Laboratories, West Groove, Pa.) at 1:5000 dilution. The blots were extensively washed with TBS-T after primary and secondary antibody incubations. Blots were developed using WesternBright ECL substrate (Advansta, Menlo Park, Calif.), following the manufacturer's instructions. Table 1 below shows the primary antibodies used to quantify the levels of different proteins by Western blot.

TABLE 1
Primary Antibodies Used for Western Blots

| | | Cataogue #; | | |
|---|---|---|---|---|
| Antibody | Source | host | Dilution | Purpose |
| Tyrosine | Invitrogen | 701949; rabbit | 1:10000; | Western blot; |
| Hydroxylase | | | 1:1000 | Immunohistochemistry |
| ChAT | Millipore | AB144P; Goat | 1:100 | Immunohistochemistry |
| phospho ERK | Cell Signaling | 9106; mouse | 1:1000; | Western blot; |
| | Technologies | | 1:200 | Immunohistochemistry |
| Total ERK | Cell Signaling | 9102; rabbit | 1:2000 | Western blot |
| | Technologies | | | |
| phospho Akt- | Cell Signaling | 13038; rabbit | 1:1000 | Western blot |
| T308 | Technologies | | | |
| phospho Akt- | Cell Signaling | 4060; rabbit | 1:1000 | Western blot |
| S473 | Technologies | | | |
| Total Akt | Cell Signaling | 2920; mouse | 1:1000 | Western blot |
| | Technologies | | | |
| phospho-p38 | Cell Signaling | 4511; rabbit | 1:1000 | Western blot |
| | Technologies | | | |
| Total p38 | Cell Signaling | 8690; rabbit | 1:1000 | Western blot |
| | Technologies | | | |
| phospho- | Cell Signaling | 9255; mouse | 1:1000 | Western blot |
| SAPK/JNK | Technologies | | | |
| Total | Cell Signaling | 9252; rabbit | 1:1000 | Western blot |
| SAPK/JNK | Technologies | | | |
| Actin | Cell Signaling | 3700; mouse | 1:5000 | Western blot |
| | Technologies | | | |
| Δ Fos B | Cell Signaling | 14695; rabbit | 1:1000; | Western blot; |
| | Technologies | | 1:100 | Immunohistochemistry |
| GRK2 | Santacruz | sc13143; | 1:500 | Western blot |
| | Biotechnology | Mouse | | |
| GRK3 | Santacruz | sc365197; | 1:300 | Western blot |
| | Biotechnology | mouse | | |
| GRK5 | R&D systems | AF4539; Goat | 1:500 | Western blot |
| GRK6 | Santacruz | sc377494; | 1:500 | Western blot |
| | Biotechnology | mouse | | |
| FoxP1 | Cell Signaling | 4402; rabbit | 1:100 | Immunohistochemistry |
| | Technologies | | | |
| Histamine H2 | Genetex | GTX108152; | 1:200 | Immunohistochemistry |
| receptor | | rabbit | | |

### Tissue Immunohistochemistry and Imaging

Immunohistochemical analysis was performed with commonly known procedures. Mice were transcardially perfused with ice-cold 4% paraformaldehyde in PBS 45 minutes after challenge with L-DOPA or DOPA-ranitidine formulations. The brain was dissected out, post-fixed in 4% PFA overnight at 4° C., and cryoprotected using 30% sucrose in PBS. Cryoprotected brains were stored at −80° C. For TH immunostaining 30 μm coronal sections were taken around both CPu and substantia nigra, to examine dopaminergic denervation at the level of striatum and neuronal cell body loss at the level of substantia nigra. For pERK, ChAT, FoxP and AFos B expression, only coronal sections at the level of striatum was used. Free-floating coronal sections were blocked in blocking buffer (PBS with 2% BSA, 0.3% TritonX-100) for 1 hr at room temperature with gentle rocking. Sections were then transferred to incubation solution (PBS with 1% BSA, 0.03% TritonX-100) and the respective primary antibody as shown in table 1 and incubated at 4° C. for 24-48 hrs with gentle shaking. The sections were mounted with fluorescein or DAPI containing mounting media and serial z stack images were taken using Zeiss laser scanning confocal microscope LSM 800 at Stanford Cell Sciences Imaging facility.

### Data Analysis

Western blots were quantified using band densitometry in Image J software (NIH version). A calibration curve was constructed for each blot from samples of known concentration and fitted using linear equations in Prism 7.0 (Graphpad Software, La Jolla, Calif.) to avoid variability among different blots. Results are represented as group mean±SEM and analyzed using one-way or repeated-measures ANOVA (RM-ANOVA), with post hoc Bonferroni tests. The mouse cylinder test was analyzed with RM-ANOVA, with group (DOPA and DOPA-ranitidine formulations) as a between-group factor and saline vs. DOPA treatment as a within-group factor. AIMs scores were analyzed using the Kruskal Wallis test for pre-testing (DOPA injected animals) and the Mann-Whitney U test for the post-testing sessions (DOPA vs DOPA-ranitidine formulations). Statistical analysis was performed using StatView (SAS Institute, Cary, N.C.).

### Example 4: Gelatinized Sustained Release Formulations of L-DOPA, DOPA Decarboxylases and Ranitidine

Methods:

Histamine antagonist ranitidine was given along with L-DOPA to determine if the combination can reverse the dyskinetic behavioral and GRK expression seen after DA depletion and subsequent L-DOPA therapy. An oral formulation of L-DOPA along with ranitidine and peripheral DOPA decarboxylases such as carbidopa or benserazide was formulated. Two different doses of the peripheral DOPA decarboxylases were used to optimize the availability of L-DOPA to the brain. The efficacy of the oral formulations was tested using the mouse cylinder test to assess the extent of akinesia relief in hemiparkinsonian mice.

Behavioral Testing.

The efficacy of the DOPA formulations to relieve the parkinsonian symptoms in the hemiparkinsonian mouse model were tested. The mouse cylinder test was used to study the effect of DOPA in relieving the akinesia seen in PD. Cylinder touches with the paw contralateral to the 6-OHDA lesion, with the digits extended to support the positioning of the body as shown in FIG. 6B, were counted and compared with the usage of the normal (ipsilateral) paw. Both DOPA and DOPA-ranitidine formulations produced significantly increased contralateral paw usage, compared to saline treatment, suggesting that all the DOPA formulations were able to relieve the akinesia seen in hemiparkinsonian mice (FIG. 6C). In FIG. 6C, usage of the paw contralateral to the 6-OHDA lesion in hemiparkinsonian mice with saline, DOPA and ranitidine+DOPA formulation treatments. Formulations IA, IB, IIA and IIB contain DOPA, peripheral decarboxylase inhibitors, and the H2 antagonist ranitidine, as described in Materials and Methods. DOPA and DOPA-ranitidine formulations showed significantly increased contralateral paw usage relative to saline treatment DOPA and DOPA-ranitidine formulations did not differ in their ability to increase contralateral paw usage (FIG. 6D).

Conclusion:

FIG. 6D illustrates an improvement in contralateral paw usage, quantified as % increase over saline treatment, did not differ between L-DOPA and ranitidine/DOPA formulations.

### Example 5: Effect of DOPA Formulations on LID in Hemiparkinsonian Mice

Background:

The effect of DOPA-ranitidine formulations on the L-DOPA induced dyskinesia in hemiparkinsonian mice was studied in this example.

Methods:

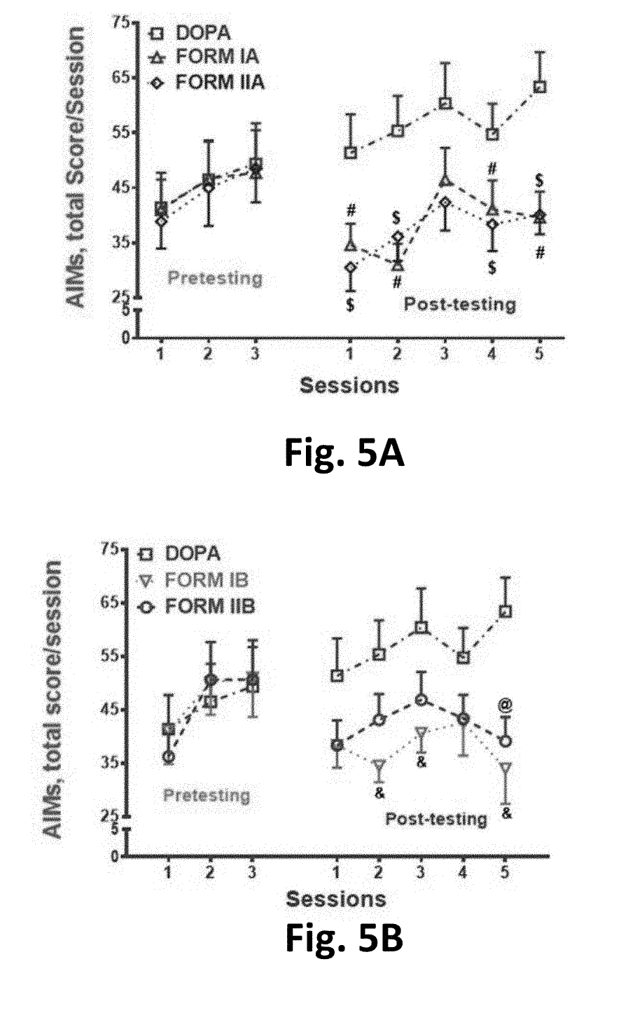

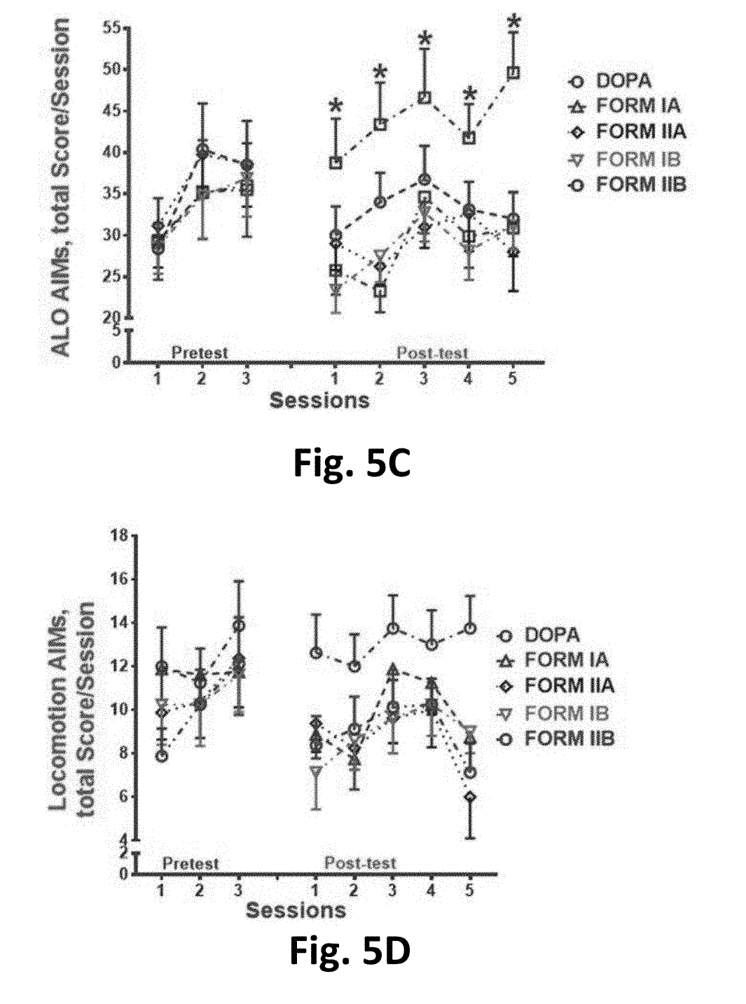

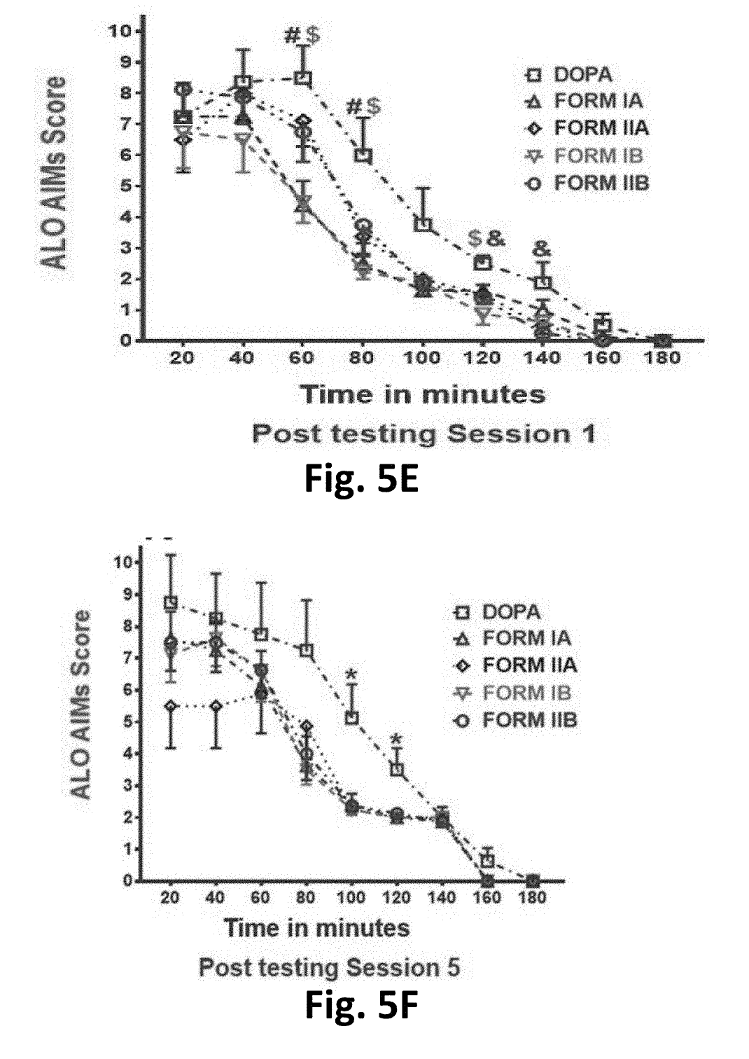

Hemiparkinsonian mice were treated for 9 days with daily injections of L-DOPA (5 mg/Kg) along with benserazide (10 mg/Kg), as reported by previous studies studying LID, to induce abnormal involuntary movements (AIMs; see FIG. 6A for experimental sequence). Dyskinetic behavior, including orolingual-forelimb dyskinesia (involuntary contralateral movements of the tongue and orofacial muscles), axial dystonia (twisting movements of the body) and locomotor/rotational behavior are shown in FIG. 6E and was quantified every third day, sampling every 20 minutes for 3 hours after L-DOPA injection; AIMs are represented as cumulative scores per session (FIG. 5A, B) and as ALO AIMs (FIG. 5C) and locomotion AIMs (FIG. 5D). Mice exhibited increasing AIMs over the course of 9 days of L-DOPA administration (FIG. 5A-D ‘pretesting’).

After 9 days of L-DOPA/benserazide, mice were separated into three groups (n=8 per group), balanced for AIMs, where the groups exhibited equivalent AIMs at the end of the pre-test period. Animals that did not show dyskinetic behaviors were excluded. Mice were rested for a week before post-testing. During post-testing, mice received daily injections of L-DOPA (mixed with benserazide) or one of the four DOPA-ranitidine formulations for 15 days; AIMs were again quantified every 20 minutes; cumulative dyskinetic scores over 3 hours are shown in FIGS. 5A-5F. While daily L-DOPA injections produced increasing AIMs, co-administration of ranitidine significantly attenuated abnormal movements, across all DOPA-ranitidine formulations (FIG. 5A-B). In FIG. 5A, total AIMs scores for ranitidine/DOPA formulations IA and IIA, which contained low-dose peripheral DOPA decarboxylase concentrations. There was a significant effect of formulation across days; post-hoc Mann-Whitney tests showed reduced AIMs after treatment with ranitidine, relative to treatment with L-DOPA alone, on all days for formulation IA and on most days for formulation IIA. In FIG. 5B, total AIMs score for ranitidine/DOPA formulations IB and IIB, which contain higher concentrations of peripheral DOPA decarboxylase inhibitors. There was a significant effect of formulation across days. Post-hoc testing showed reduced AIMs after treatment with formulation IB on sessions 2, 3 and 5 and after treatment with formulation IIB on session 5.

The total AIMs were further analyzed into ALO AIMs (FIG. 5C) and locomotion AIMs (FIG. 5D). FIG. 5C shows the ALO AIMs scores for the pre and post-testing sessions in all the groups. The ALO AIMs showed that the ranitidine containing DOPA formulations had significantly reduced ALO AIM scores than the animals that received control DOPA. The data for the ALO AIMs containing (Oro-lingual, Forelimb and Axial dystonia) were separated from the locomotion or rotational AIMs to have a better understanding. FIG. 5D shows locomotion AIMs in the pre and post testing session is represented. As far as the locomotion AIMs were concerned, the effect was somewhat attenuated in ranitidine DOPA formulations. Although the control DOPA groups had higher locomotor/rotational movements than the ranitidine groups though not significantly different (FIG. 5D). The dynamics of the AIMs during the testing period were of interest and the ALO AIMs plotted over 20 minutes bins for post testing sessions 1 and 5 to see the dyskinetic behavior. As anticipated the control DOPA groups had higher ALO AIMs in both the testing sessions with the ranitidine/DOPA formulations having lesser AIM scores and earlier complete attenuation times than the control DOPA groups (FIG. 5A-B). Interestingly, the ranitidine/DOPA formulations with benserazide had significantly lesser attenuation times than carbidopa in the post testing session 1 (FIG. 5A). In the post testing session 5, the attenuation times for the ranitidine/DOPA formulations shifts to the right significant reductions in ALO AIM scores only between 100 and 120 mins (FIG. 5A) could be observed after which all the groups showed similar dyskinetic behavior.

FIG. 5E shows a representation of ALO AIMs in 20 mins bin during the post testing session 1 in the groups. The formulations IA and IB with the DOPA decarboxylase carbidopa had significantly lower ALO AIMs score (Bonferroni posthoc DOPA vs Form IA p<0.0019; DOPA vs Form IB p<0.0026 for 60 mins and DOPA vs Form IA p<0.0044; DOPA vs Form IB p<0.0025 for 80 mins) during the earlier and later stages of the ALO AIMs scoring and the formulation with the DOPA decarboxylase benserazide had lower ALO AIMs scores at the later time points than the control DOPA group (Bonferroni posthoc DOPA vs Form IB p<0.0001; DOPA vs Form IIB p<0.0040 for 120 mins and DOPA vs Form IIB p<0.0046 for 140 mins).

FIG. 5F shows a representation of ALO AIMs in 20 mins bin during the post testing session 5 in the groups. In the post testing session 5, the significance in the ALO AIMs between the control DOPA and the formulations groups shifted to the right. All the formulation groups had significantly less ALO AIMs scores in the 100 min time point (p<0.0007, 0.0004, 0.0004 and 0.0007 between control DOPA and Form IA, IIA, IB, IIB respectively) and 120 mine time point (p<0.0016, 0.0016, 0.0016 and 0.0034 between control DOPA and Form IA, IIA, IB, IIB respectively).

Conclusion:

Nevertheless, the ranitidine/DOPA formulations effectively relieved the akinesia seen in PD mice similar to control DOPA and at the same time reduced the dyskinetic behavior significantly as shown by ALO AIMs.

### Example 6: Effect of Ranitidine on the Levels of G Protein Coupled Receptor Kinases (GRKs) in the Brain

Methods: There are 7 GRK subtypes, of which 4 are expressed in the brain. GRK6 is expressed at the highest level in the caudate-putamen. One of the marked effects of DA depletion in PD is the downregulation of GRK3 and GRK6 in the lesioned striatum; this effect is not reversed by DA supplementation therapy with L-DOPA based on previous observations. The restoration of GRK expression might underlie the reduced AIMs seen after ranitidine treatment.

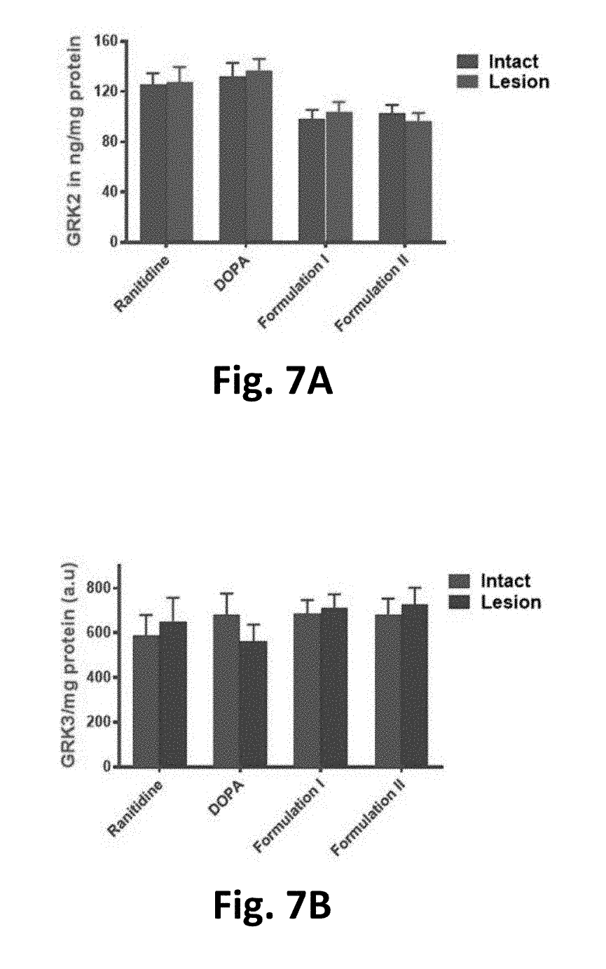

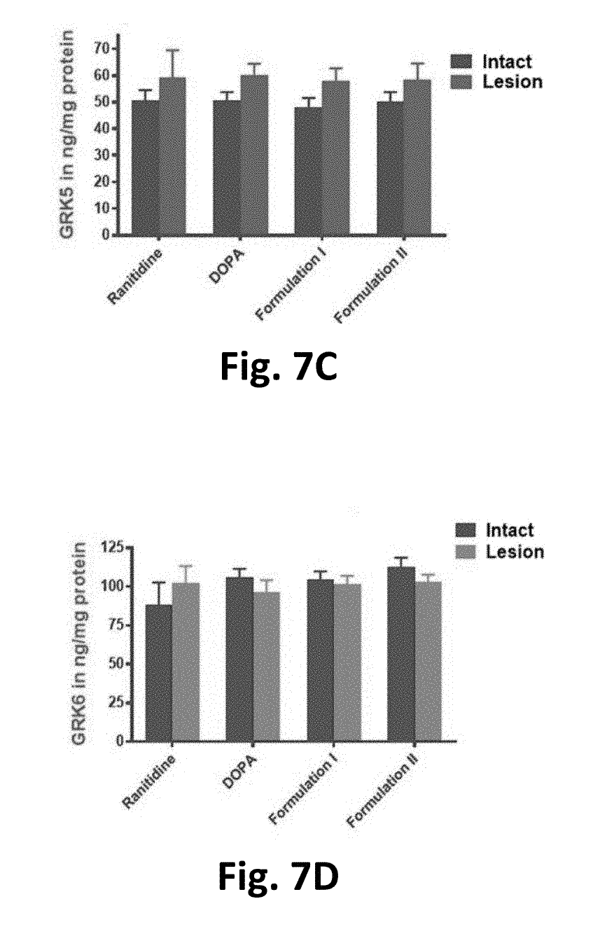

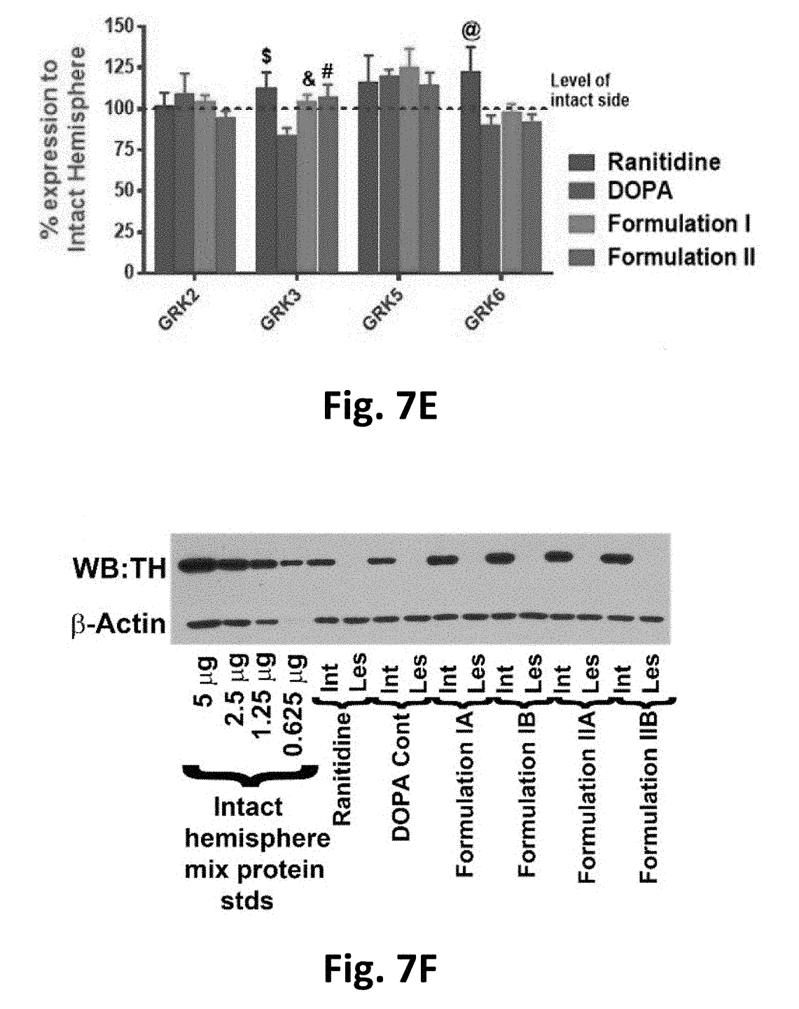

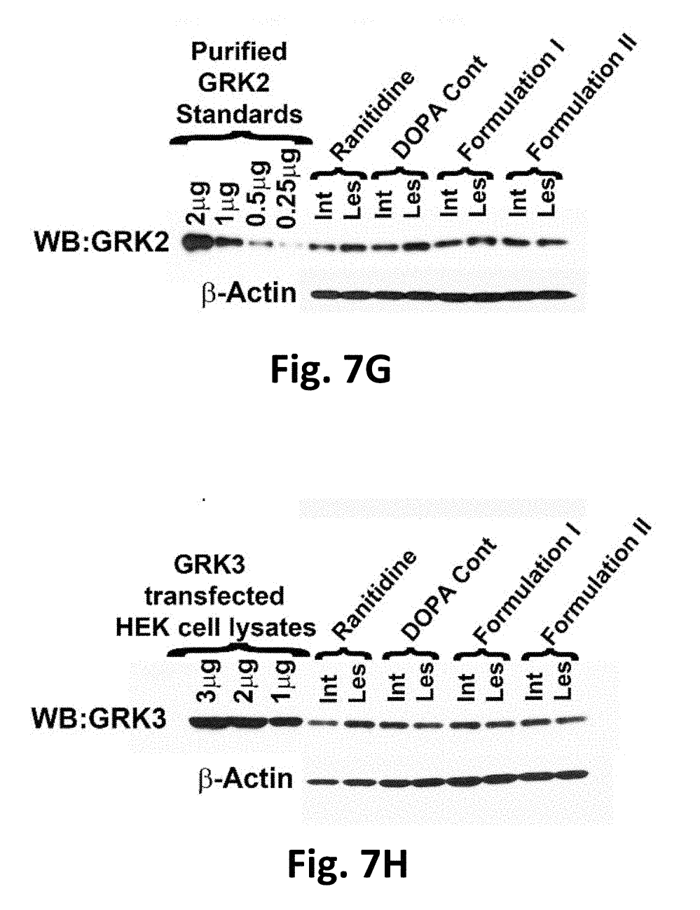

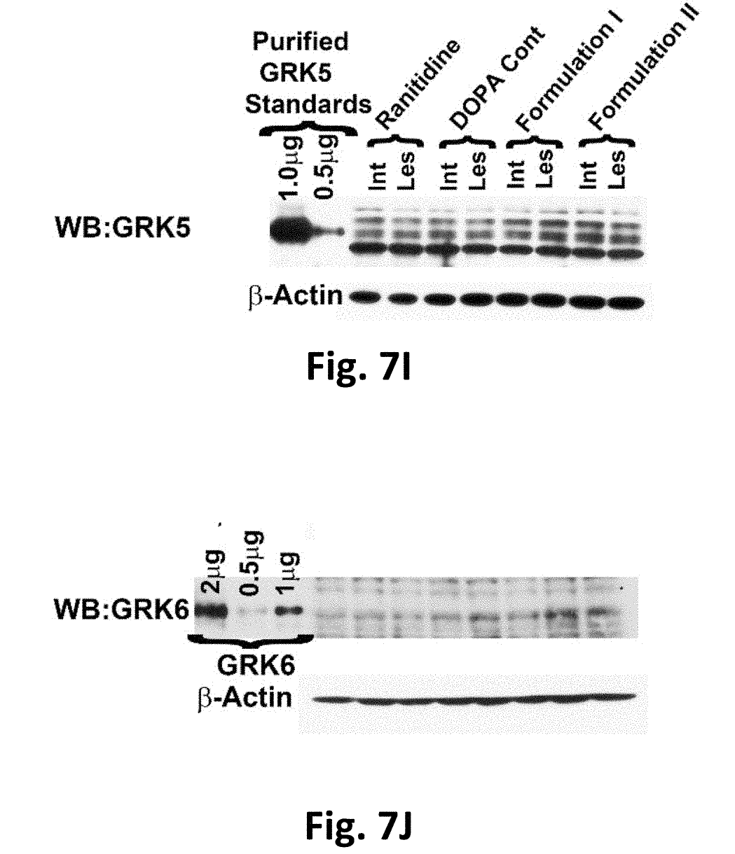

GRK2, GRK3, GRK5 and GRK6 were quantified in the intact and lesioned striatum of hemiparkinsonian mice. For analysis, animals treated with DOPA-ranitidine formulations IA and IB (low and high dose of peripheral DOPA decarboxylase inhibitor carbidopa; FIG. 5A) were pooled with those treated with formulations IIA and IIB (low and high dose peripheral DOPA decarboxylase inhibitors benserazide; FIG. 5B). No statistically significant differences in the expression of GRK2, GRK3, GRK5 and GRK6 levels were observed between the intact and the lesioned striatum in any experimental groups as analyzed by repeated measure ANOVA (RM-ANOVA) (FIGS. 7A-D). FIGS. 7A-D show graphical representations of the quantitation of GRK2 (2A), GRK3 (2B), GRK5 (2C) and GRK6 (2D) in the intact and lesioned hemispheres of the brain from ranitidine, DOPA, and ranitidine/DOPA formulation groups as measured by western blotting at the level of striatum. Total GRK2 was nominally reduced on both sides in DOPA-ranitidine formulation treated mice relative to mice treated with ranitidine or DOPA alone, though this effect did not reach statistical significance (FIG. 7A). A different approach was used to study the difference in the expression of GRKs between the intact and lesioned hemispheres after normalization of the expression to a house keeping protein such as actin and plotted the difference between intact and lesioned hemispheres as a percentage expression to the intact side (FIG. 7E). The dotted line in FIG. 7E represents the level (at 100%) of the corresponding GRKs on the intact side. The differences in expression for the GRKs on the lesioned side in the experimental groups are plotted. FIG. 7E showed that GRK3 was significantly reduced in the lesioned hemisphere in the DOPA control group (FIG. 7E), as has been reported previously; but expression was normalized after treatment with either ranitidine or DOPA-ranitidine formulations (F=3.908, p<0.0171, N=5 (Ranitidine), N=9 (DOPA), N=12 (Formulation I) and N=11 (Formulation II) as per the % expression from the intact side). Percent decrease in GRK3 in the lesioned hemisphere as per Bonferroni post-hoc analysis was significantly attenuated in the ranitidine (p<0.0078) and formulation II groups (p<0.0072) relative to the DOPA-treated group; attenuation in the formulation I group was numerically similar but did not survive Bonferroni correction (uncorrected p<0.0155). No changes were seen in GRK2 and the lesioned side had higher expression of GRK5 in all the experimental groups (FIG. 7E). GRK6 was a real puzzle as the results contradicted the previously published data. Only a marginal degree of down regulation of GRK6 in the DOPA and ranitidine/DOPA groups was observed in the lesioned hemisphere than the intact control side (FIGS. 7D, 7E). This contradiction in GRK6 expression could be explained by the fact that the observed changes in this study were in mice rather than rats where consistent difference in GRK6 expression was observed in dopamine (DA) depleted side and the other reason could be with the detection range of the antibody which was used for the western analysis. A mouse monoclonal antibody was used against GRK6 from Santacruz. GRK6 is a serine threonine kinase with 3 splice variants (GRK6A, GRK6B and GRK6C) and previous studies with the analysis of GRK6 has shown that GRK6 antibody detects the different isoform of GRK6 from the same firm. The effect of DOPA on the expression of GRK6 in the mouse model was not predictable as there is little information regarding this in hemiparkinsonian mice model.

The confirmation of DA depletion in hemiparkinsonian mice was determined by quantifying TH in the intact and the lesioned hemispheres (FIG. 7F). FIG. 7F shows an image of a western blot showing TH expression in the intact vs lesioned hemisphere of the brain samples from this study groups along with β-actin control. TH was completely lost in the lesioned side indicating complete loss of functional dopaminergic input in to the striatum. Animals with detectable TH in the lesioned hemisphere were excluded from all analyses. The representative blots of all GRKs are shown in FIGS. 7G-J along with β-actin loading controls below each GRK. Specifically, FIG. 7G shows a representative western blot for GRK2 along with β-actin control; FIG. 7H shows a representative western blot for GRK3 along with β-actin control; FIG. 7I shows a representative western blot for GRK5 along with β-actin control; and FIG. 7J shows a representative western blot for GRK6 along with β-actin control. The blots were run to show the expression profile of GRKs in the different groups to represent the graphs.

Conclusion:

In aggregate, these data suggest that GRK3 down regulation in hemiparkinsonian mice is reversed by ranitidine treatment, paralleling the mitigation of dyskinesia produced by chronic DOPA therapy.

### Example 7: Effect of Histamine Depletion on the Expression of G Protein Coupled Receptor Kinases (GRKs) in the Brain

Methods:

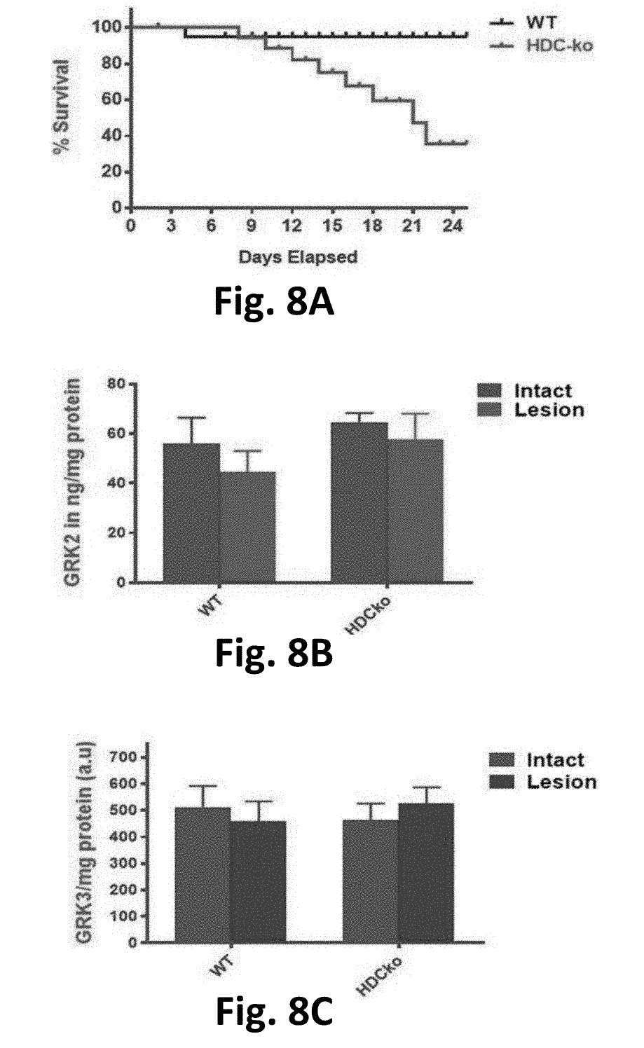

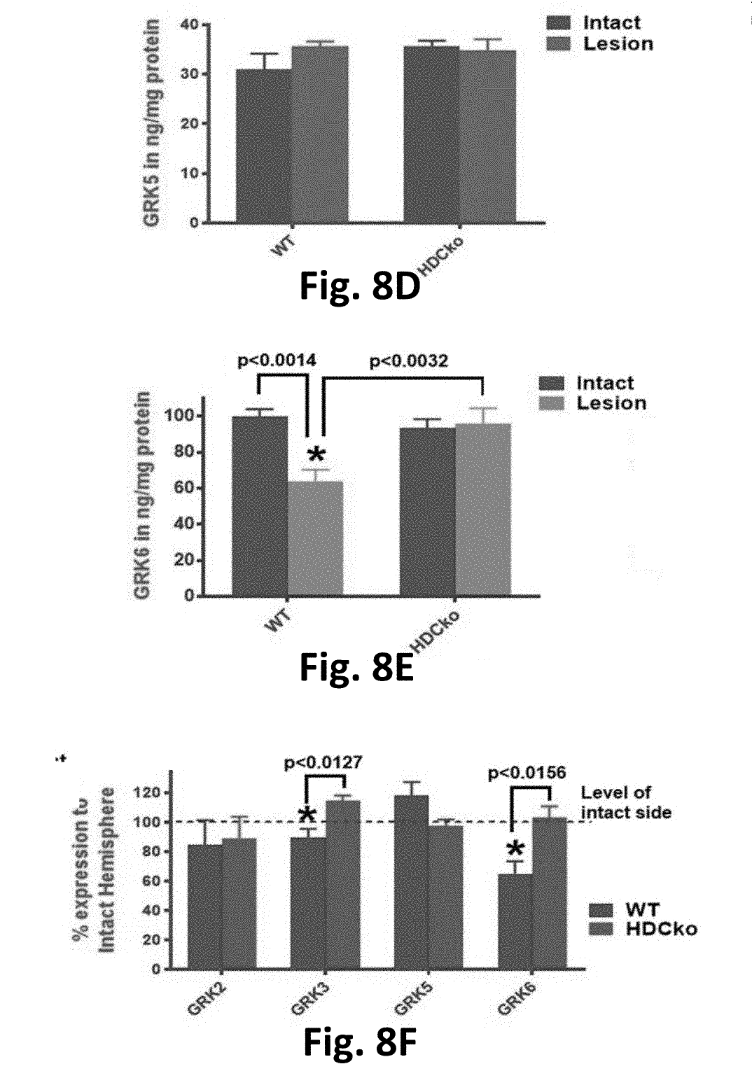

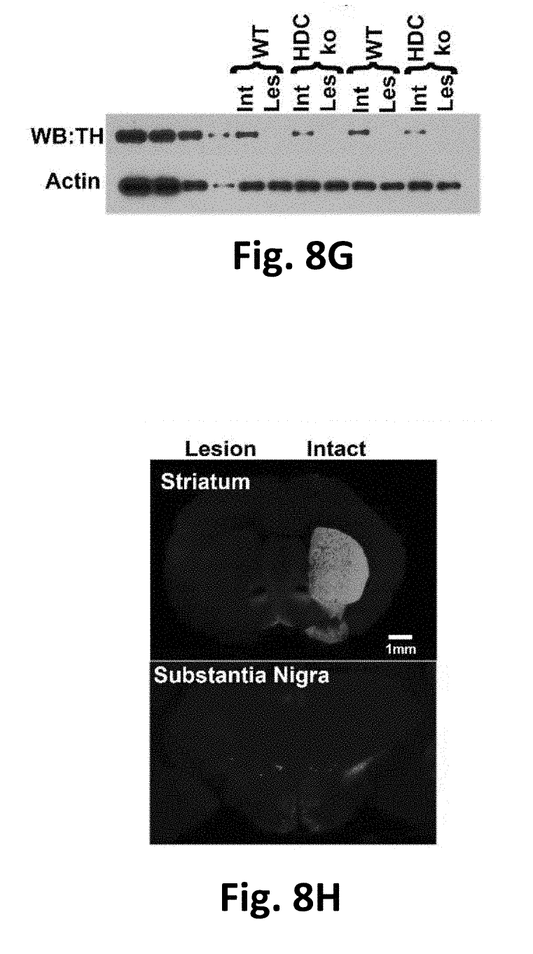

FIG. 8A shows percentage mice survival in histamine-deficient mice. Histidine decarboxylase (HDC) catalyzes the conversion of histidine to histamine; Hdc knockout mice have little or no histamine in the brain. histamine and dopamine interact in their regulation of activity and signaling in the basal ganglia. 6-OHDA lesions in mice results in significant morbidity due to reduced food intake during the initial weeks post-surgery; lesioned mice are supplemented with a liquid diet to sustain the initial weeks of surgery. 6-OHDA was infused unilaterally into the striatum in Hdc-KO mice and wild-type littermates. A significant mortality in Hdc-KO mice was observed despite this supplementation, compared with their wt littermates (FIG. 8A). Both histamine and dopamine activate GPCRs, which regulate GRKs in the striatum. This genetic knock out model was used to mitigate the effect of loss/down regulation of histamine on the expression profile of GRKs in the dopamine depleted striatum. By inducing parkinsonism in Hdc-KO mice, the consequences of dual loss of histamine and dopamine on the expression of GRKs was investigated. FIGS. 8B-E show the expression profile of GRKs in Hdc-KO mice and their wt littermates. Specifically, FIG. 8B shows levels of GRK2 protein; FIG. 8C shows levels of GRK3 protein; FIG. 8D shows levels of GRK5 protein; and FIG. 8E shows levels of GRK6 protein. GRK6 protein levels were reduced on the 6-OHDA lesioned side in wild-type animals (F=7.124, N=4, p<0.0053) and by Bonferroni post-hoc analysis between the intact and lesioned hemisphere of WT (p<0.0014) but not in Hdc-KO (WT vs. KO lesioned side: p<0.0032). Similar to previous analysis of GRKs in the ranitidine/DOPA formulations, GRKs in wt and Hdc-KO mice was represented as percent expression to intact hemisphere (FIG. 8F). DA depletion, as in the 6-OHDA lesion model, causes irreversible loss of GRK3 and GRK6; this loss is not reversed by DOPA therapy in the rat 6-OHDA model, but it is reversed by reduction of histamine in the Hdc-KO mice (FIG. 8F). FIG. 8F shows percent change in GRK protein; relative to the intact hemisphere; GRK3 and GRK6 were reduced in the lesioned hemisphere of WT mice but not of Hdc-KO mice (WT vs Hdc-KO: F=12.33, N=4, p<0.0127 and 0.0156 for GRK3 and GRK6 respectively).

Conclusion:

In wt mice, the levels of GRK2, 3 and 6 were reduced in the 6-OHDA-lesioned hemisphere whereas GRK5 was increased slightly (FIG. 8B-F) mirroring the effect seen with ranitidine/DOPA formulations; the interaction for GRK6 compared by 2-way ANOVA for group (WT vs Hdc-KO) and protein (GRK6) was significant (F=7.124, N=4, p<0.0053) this reduction reached statistical significance in the case of GRK6 between the intact and lesioned hemisphere in WT by bonferroni posthoc analysis (p<0.0014). In Hdc-KO mice, on the other hand, there was no loss of GRK6 in the lesioned hemisphere and the difference between the lesioned hemispheres of Hdc-KO and WT littermates were significant by Bonferroni post-hoc analysis (p<0.0032, wt vs Hdc-KO FIG. 8E). This was confirmed when the levels of GRKs were normalized to those seen in the intact hemisphere in each animal: GRK3 and 6 were decreased in WT animals, and this loss was not seen in Hdc-KO mice in the lesioned hemisphere when the % expression from the intact side (p<0.0127 and 0.0156 for GRK3 and 6 respectively as compared by ANOVA with posthoc Bonferroni analysis, FIG. 8F) was considered. The reason that significant reductions in GRK3 and GRK6 were observed could be due to the fact that these mice were drug naïve and were not exposed to DOPA treatments and perhaps DOPA treatment in ranitidine formulation groups might have compromised the expression profile of GRK3 and GRK6 in the similar age group mice. Lesions were confirmed by TH western blot analysis for expression in intact and lesioned hemispheres of the Hdc-KO and wt littermates along with (3-actin control (FIG. 8G) and immunostaining for tyrosine hydroxylase (TH), which was almost completely lost on the lesioned side (FIG. 8H) at the level of striatum (TH fibers) and Substantia nigra (neuronal cell body). In FIG. 8H, the loss of TH fibers and cell body respectively in the lesioned side, and the scale bar represents 1 mm. TH levels were similarly reduced in Hdc-KO mice and their wt littermates as detected by western blot analysis and normalized to β-actin levels. The expression profile of GRKs in the Hdc-KO mice and their wt littermates are shown in FIG. 8I.

### Example 8: Signaling Changes after 112 Antagonism in DOPA-Treated Hemiparkinsonian Mice

Background:

ERK activation by DOPA therapy in the DA depleted brain has been implicated in the occurrence of dyskinesia. Acute DOPA treatment results in supersensitive ERK responsivity; this is deactivated by chronic DOPA treatment.

Methods:

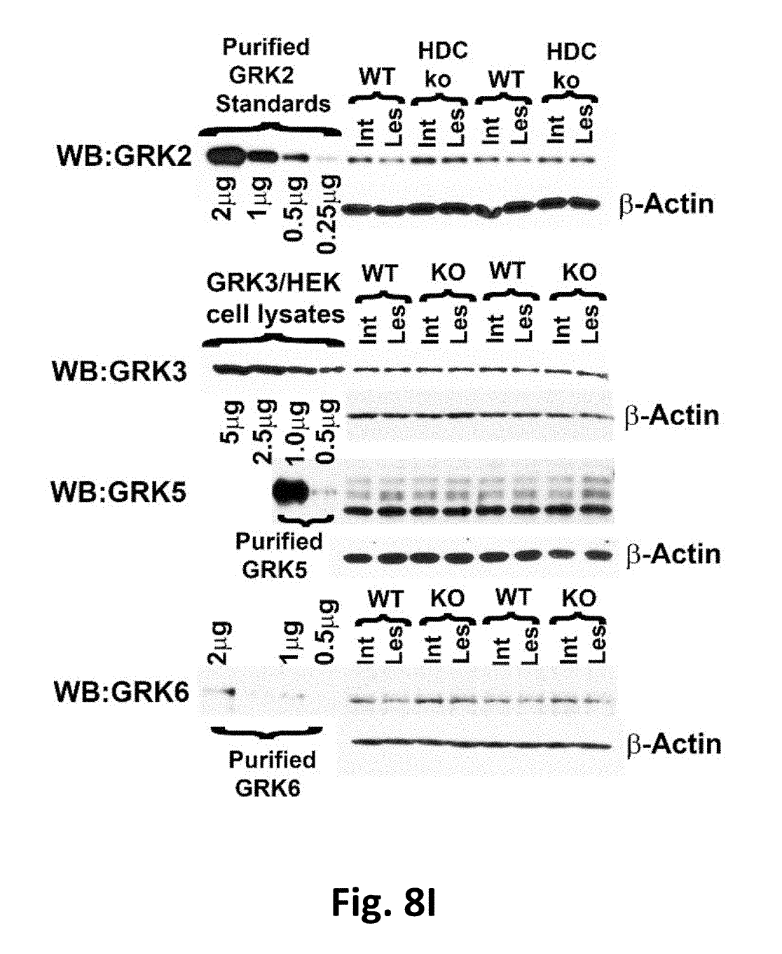

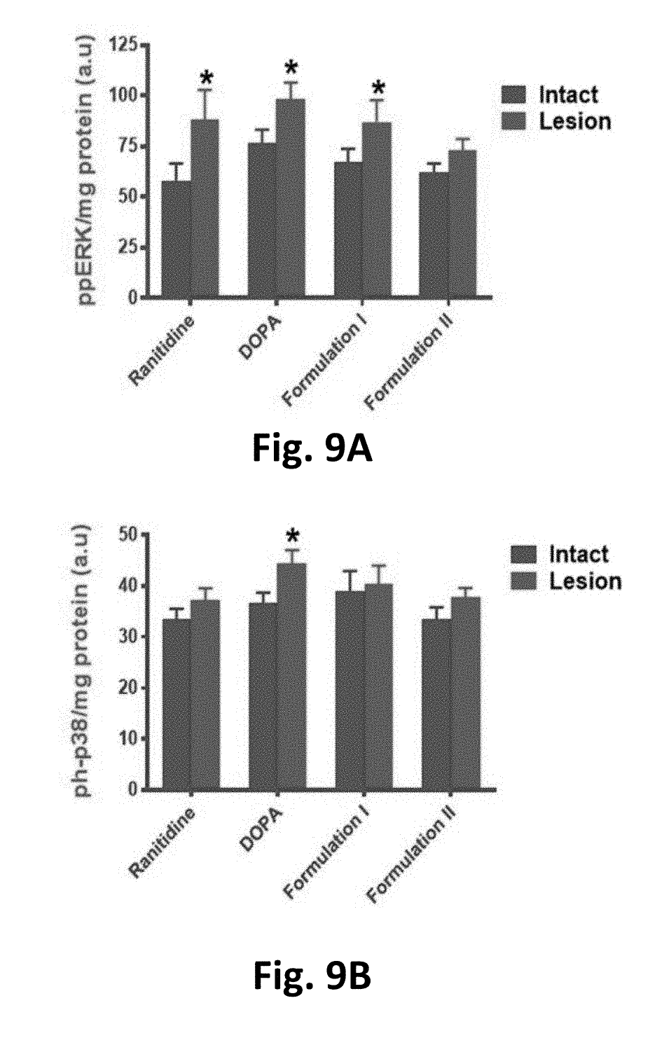

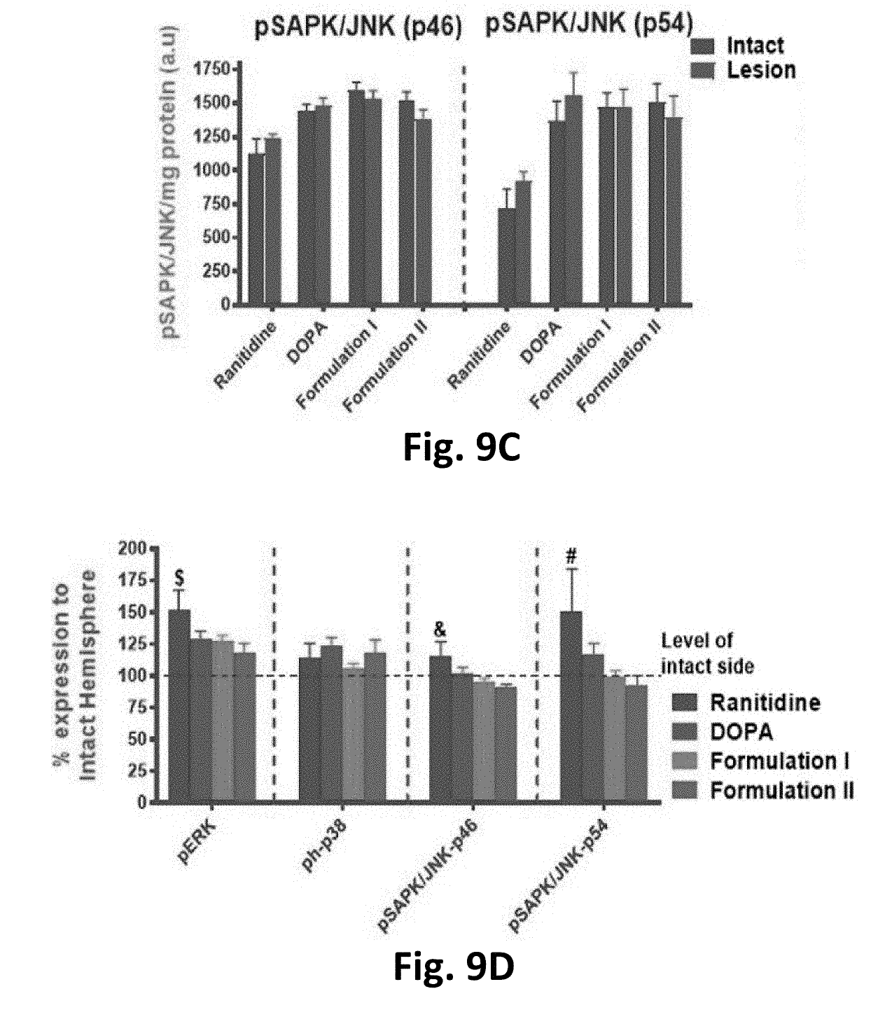

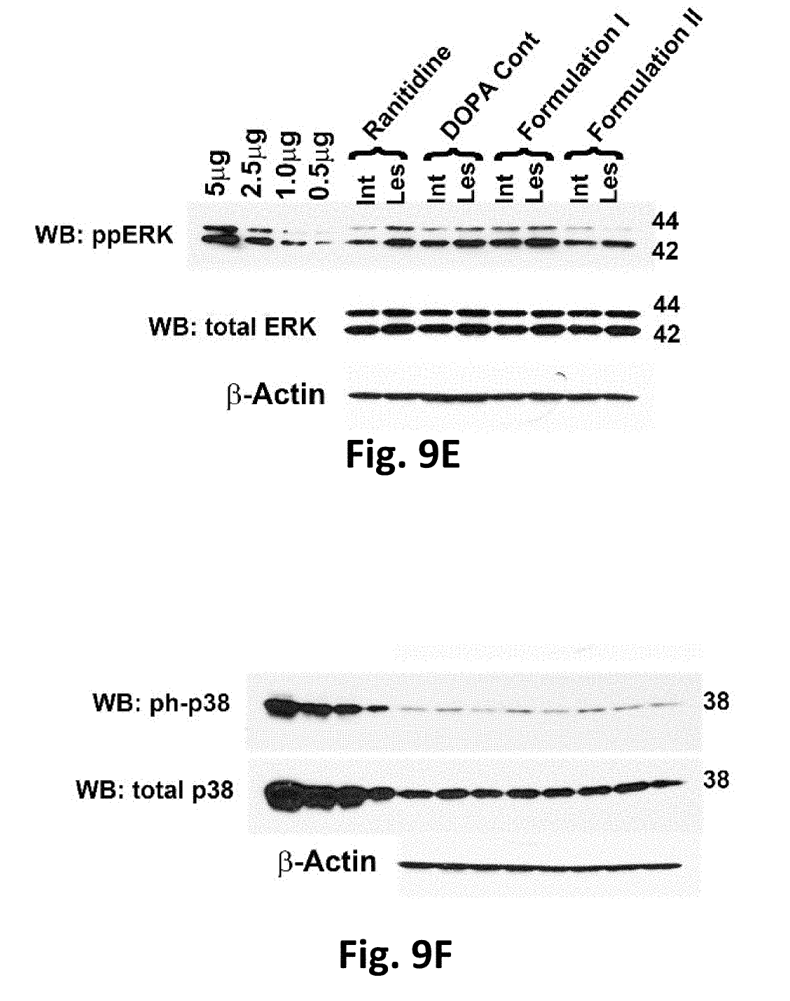

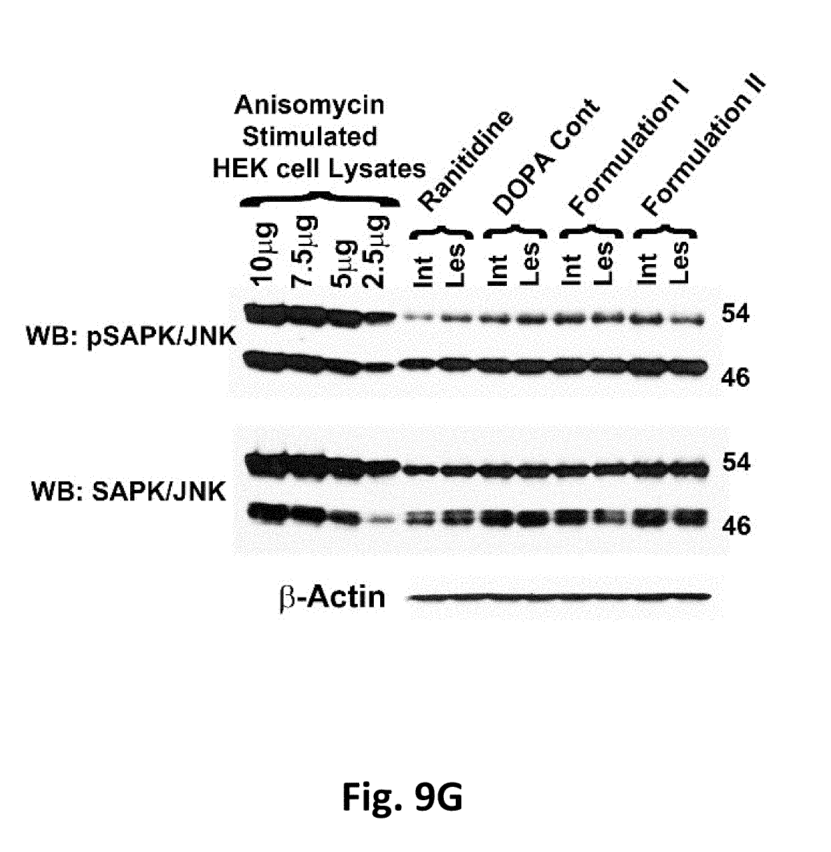

ERK was activated by treatment with L-DOPA or DOPA-ranitidine formulations, though this effect was attenuated in mice treated with formulation II (higher dose peripheral dopa decarboxylase inhibitors). FIGS. 9A-9G show phosphorylation of components of the MAPK signaling pathway in hemiparkinsonian mice after treatment with ranitidine, L-DOPA, or DOPA-ranitidine formulations. Specifically, FIG. 9A shows the quantitation of the ppERK in the intact and lesioned hemispheres of the experimental groups. ERK phosphorylation was increased in the lesioned hemisphere after treatment with ranitidine, L-DOPA, or DOPA-ranitidine formulation I; FIG. 9B shows western blot quantitation of phospho p38 in the intact and lesioned hemispheres of the striatum. p38 phosphorylation was increased in the lesioned hemisphere only after DOPA treatment; FIG. 9C shows levels of phospho SAPK/JNKs p46 and p54 in the intact and lesioned hemispheres of the experimental groups, where no significant difference in pSAPK/JNK levels in any of the groups was observed; FIG. 9D shows phosphorylation and expression data from FIGS. 9A-9C expressed as % expression in the lesioned side relative to the intact side. The dotted line indicates the levels of the MAPK proteins in the intact side represented at 100% level; and FIGS. 9E-9G show sample western blots for analysis of the MAPK signaling pathway, where FIG. 9E shows ppERK and total ERK, FIG. 9F shows phospho p38 and total p38, and FIG. 9G shows phospho SAPK/JNK and total SAPK/JNK.

Surprisingly, ranitidine alone also activated ERK in the lesioned hemisphere (FIGS. 9A, 9E). There was no change in total ERK levels in the lesioned hemisphere in any of the experimental groups. p38 was increased in the lesioned hemisphere after DOPA treatment, but not in the other experimental groups (FIGS. 9B, 9F). This effect was no longer seen when data were normalized to expression levels from the intact hemisphere in the different experimental groups with ranitidine/DOPA formulations (FIG. 9D). There was no change in the levels of total p38 in the lesioned hemispheres in any of the experimental groups.

Conclusion:

There are 3 major isoforms of JNK, JNK1, 2 and 3; each isoform has 2-4 splice variants, resulting in a total of 10 JNK isoforms. The levels of phospho JNK are shown in FIG. 9C and the corresponding percentage expression of pJNK in the lesioned side from the intact hemisphere is shown in FIG. 9D. In Western blots (FIG. 9G), two prominent bands were apparent: p46 and p54. JNK was elevated after treatment with either DOPA or any of the DOPA-ranitidine formulations in both the intact and the lesioned hemispheres, with no differences between hemispheres. However, the phosphorylation of p54 was reduced in both the intact and lesioned hemispheres of ranitidine alone group than the other ranitidine/DOPA formulation groups (FIG. 9C), the levels of total JNK 46 and JNK 54 were similar across all the experimental groups.

### Example 9: Signaling Changes of the Akt Pathway and Δ Fos B

Methods:

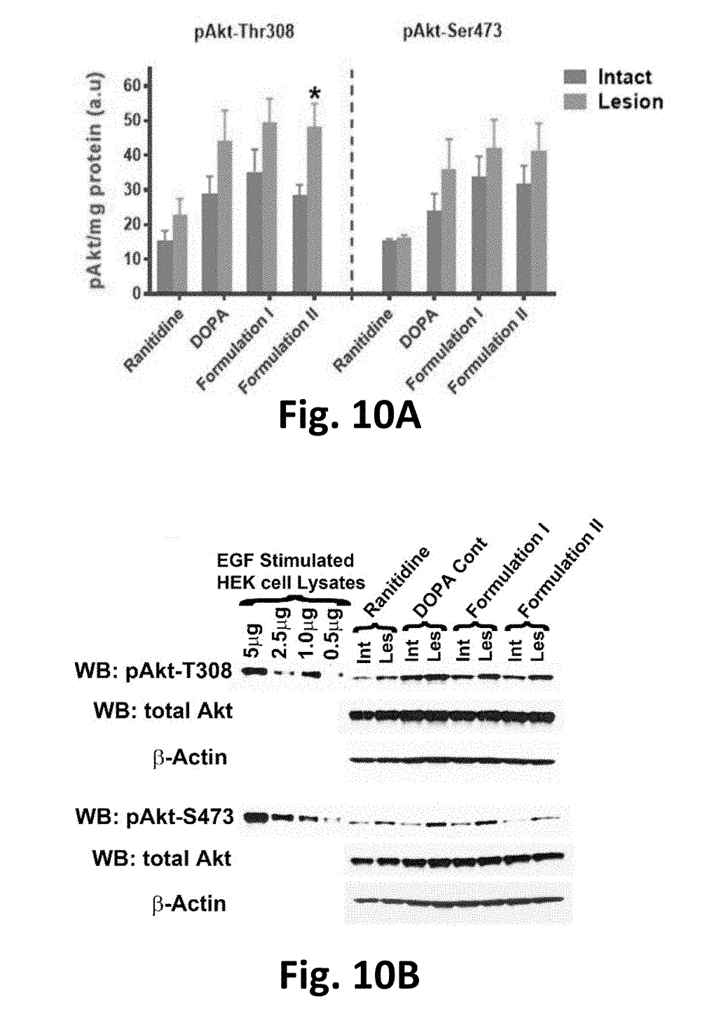

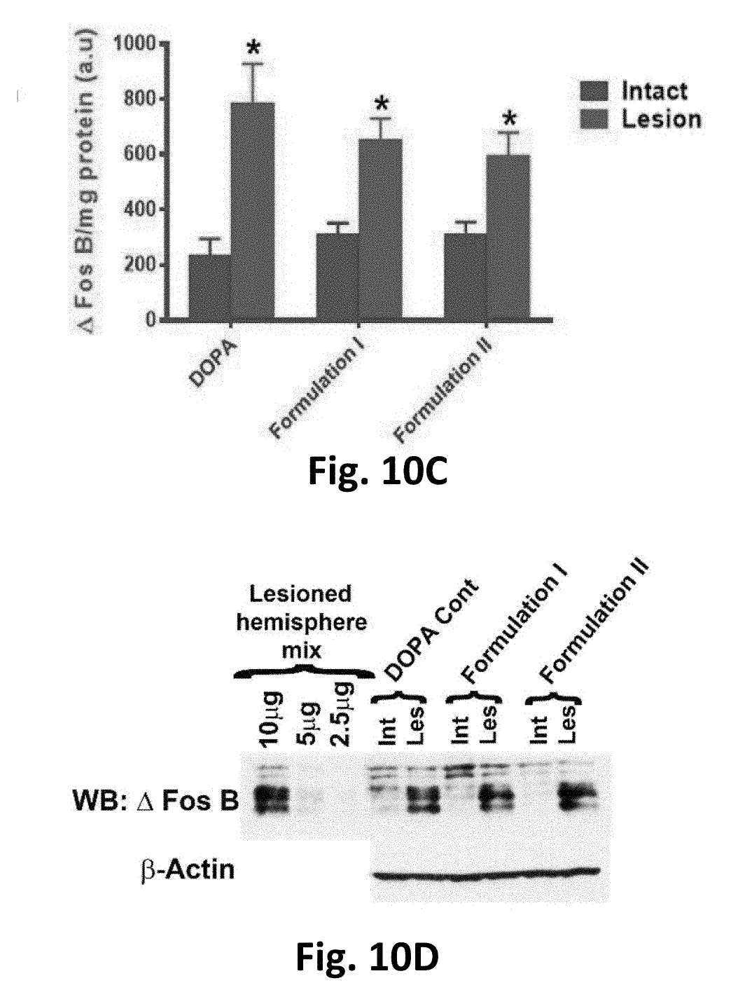

Supersensitive Akt response, in particular phosphorylation at Thr308, is implicated in dyskinesia in animal models of PD. This was confirmed in all three DOPA-treated groups, with no statistically significant difference between DOPA and DOPA-ranitidine formulation groups (FIGS. 10A and 10B). Specifically, FIG. 10A shows a graphical representation of quantitation of phospho Akt at the major (T308) and minor (S473) phosphorylation sites in the experimental groups. Akt phosphorylation at Thr308 was elevated in the lesioned striatum after L-DOPA treatment, with or without ranitidine. There was no effect of ranitidine alone. Nominal effects on phosphorylation at Ser473 were not statistically significant, while FIG. 10B shows western blot images for pAkt-T308 and pAkt-5473 along with total Akt and β-actin loading controls. There was no activation of Akt in the ranitidine control group. Nominal effects of DOPA treatment on phospho-Ser473 were not statistically significant.

Δ FosB has been shown to accumulate in the DA-depleted brain with repeated DOPA administration. Fos B accumulation correlates well with the development of dyskinesia in animal models of PD. This was confirmed: Δ FosB was increased in the DOPA-treated animals in the lesioned striatum, with no difference between animals treated with L-DOPA alone or with DOPA-ranitidine formulations. There were no effects of ranitidine alone on Δ FosB levels (FIGS. 10C and 10D). Specifically, FIG. 10C shows the quantitation of Δ Fos B accumulation in the lesioned hemisphere of the brain in the experimental groups. Δ Fos B accumulation was significantly increased on the lesioned side after L-DOPA treatment, with or without ranitidine; there was no difference between groups, while FIG. 10D shows representative western blots for Δ Fos B.

Ranitidine increased phospho-ERK in the lesioned striatum (FIG. 9A). Histaminergic input has been described on both striatal medium spiny neurons (MSNs) and ChAT-positive cholinergic interneurons (CINs). Some studies have suggested that the development of dyskinesia corresponds to a switch in the activation of ERK from MSNs to CINs. Blocking H2R with an antagonist such as ranitidine might reduce the firing of the H2R-positive neurons in the striatum and thereby dampen the activity-dependent signaling upregulated by DA replacement therapy. Immunohistochemistry was used to test this prediction.

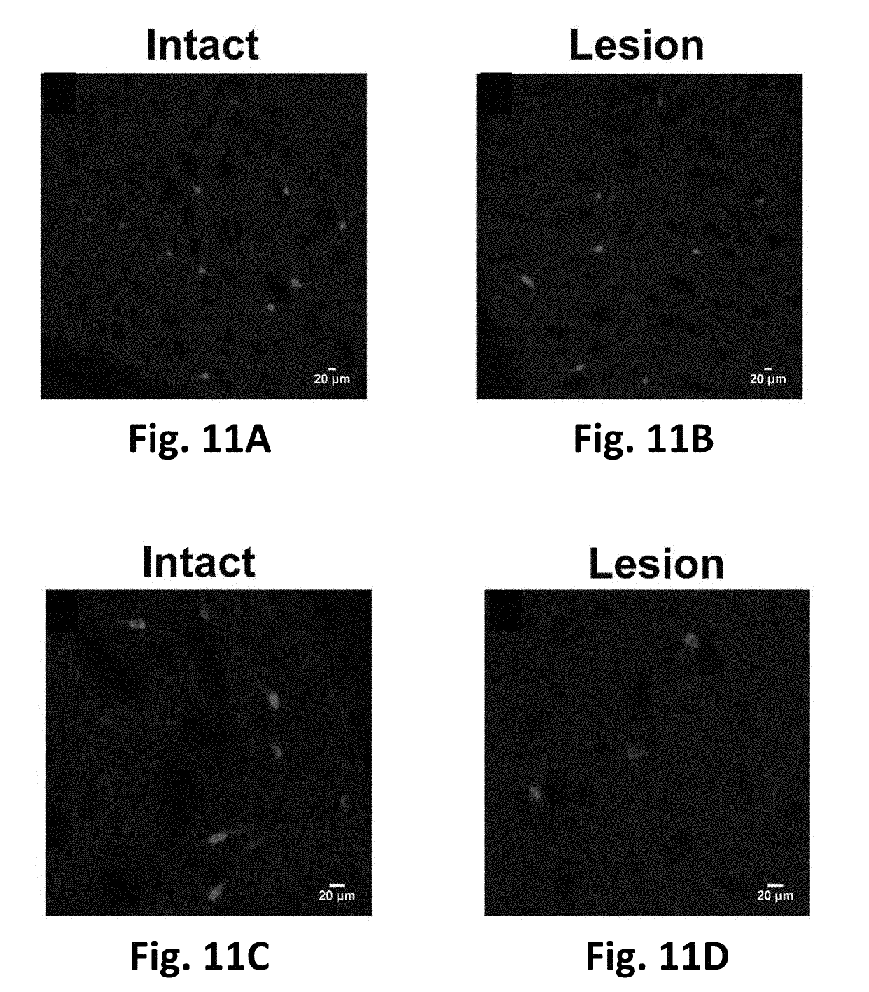

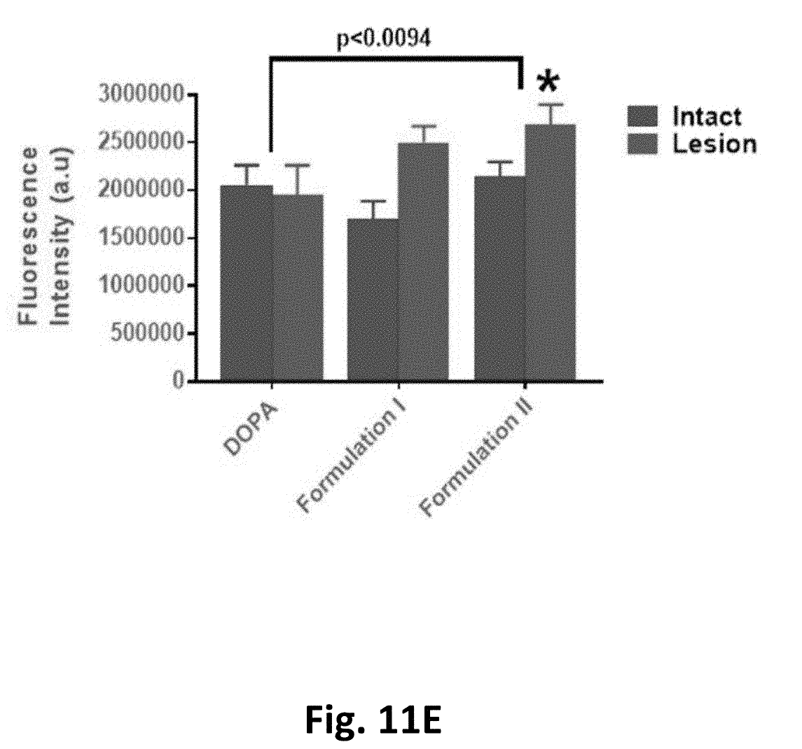

The ChAT immunoreactivity in the CINs in the intact and lesioned side of DOPA and Ranitidine/DOPA formulation groups is shown in FIG. 11A-D, which show images for ChAT positive interneurons. Specifically, these figures show immunohistochemical localization of ChAT positive interneurons in the intact and lesioned side of the brain. FIGS. 11A-11B show immunohistochemical localization at 10× magnification, while FIGS. 11C-11D show immunohistochemical localization at 20× magnification. The fluorescence intensity (FI) quantified was based on reported methods and shown in FIG. 11E. The ChAT immunoreactivity was slightly lower on the lesioned side than the intact side in the DOPA treated animals (FIG. 11E) whereas in the Ranitidine/DOPA formulation animals the FI was higher in the lesioned side than the intact side. The FI of ChAT in the lesioned side of formulation II group was significantly higher than the DOPA control groups with RM-ANOVA with posthoc Bonferroni analysis (p<0.0094).

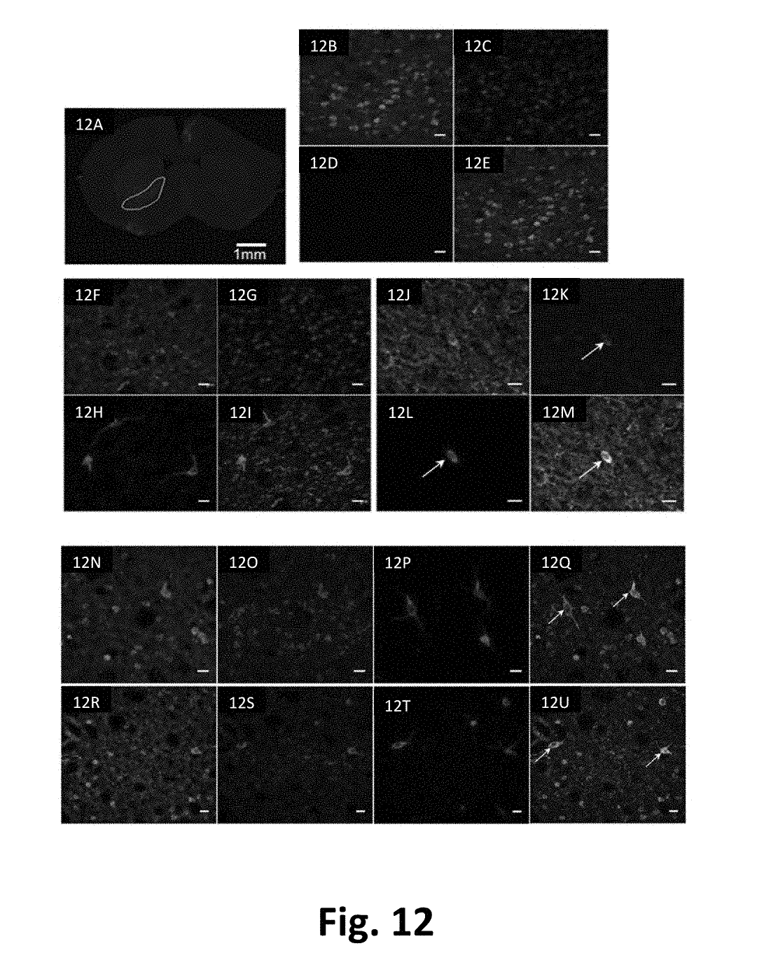

FIGS. 12A-12M show the immunohistochemical co-localization of pERK and H2R in FoxP1-positive MSNs and CINs in DOPA treated hemiparkinsonian mice. Specifically, pERK is illustrated in FIGS. 12B, 12F, AND 12J; FoxP1 is illustrated in FIGS. 12C, 12G, and 12K; and ChAT is illustrated in FIGS. 12D, 12H, and 12L. pERK was co-localized in FoxP1 positive MSNs (FIGS. 12E, 12I, and 12M), however FoxP1 staining is not seen in ChAT positive large cholinergic interneurons (FIGS. 12F-12I). H2R expression is seen in both MSNs and ChAT positive neurons (FIGS. 12J-12M). Arrow indicates co-localization. Scale bar represents 20 μm.

FIGS. 12N-12U show immunohistochemical localization of pERK, H2R in ChAT positive interneurons in ranitidine formulation treated hemiparkinsonian mice. pERK (FIGS. 12N and 12R), H2R (FIGS. 12O and 12S), ChAT (FIGS. 12P and 12T) signal in the striatum of the lesioned side. More pERK co-localization in H2R positive ChAT (FIGS. 12Q and 12U), cholinergic interneurons could be seen in the ranitidine DOPA formulation groups. Arrow indicates co-localization. Scale bar=20 μm.

pERK was elevated in the lesioned striatum, consistent with FIG. 12A. pERK was more prominent in the ventromedial and ventrolateral striatum in the DOPA-treated animals, whereas Fos B was more prominent in the dorsomedial and dorsolateral striatum (FIG. 12A). pERK seemed to be more prevalent in the MSNs (identified by immunostaining for FoxP1) than in CINs after DOPA treatment (FIGS. 12F-12I). Occasional triple labeling of pERK, ChAT and H2R in the DOPA control group was observed (FIGS. 12J-12M); this was more prominent in mice treated with DOPA-ranitidine formulations (FIGS. 12N-12U). Fos B was seen in MSNs in the dorsomedial and dorsolateral striatum, and less prominently in the ventromedial striatum.

The expression pattern of pERK and Fos B was mapped in the rostro-caudal direction in DOPA-injected animals. The immunohistochemical studies shows that the expression pattern of pERK and fos B are distinct and therefore suggests that they are regulated by different mechanisms.

Conclusion:

It is demonstrated here that ranitidine acts as a GRK3 up regulating agent. Ranitidine is one of the HA H2R antagonist and ameliorates the dyskinesia when given along with DOPA in a sustained release formulation. Previous mechanistic signaling studies with lentivirus mediated overexpression of GRK6 in rat hemiparkinsonian model had implicated that the MAPK, Akt pathways and Δ Fos B accumulation played crucial roles in the development of dyskinesia. In the knockout mouse line, the Hdc-KO mice which were deficient in the rate limiting enzyme histidine decarboxylase that converts histidine to histamine, the HA is constitutively absent in the brain which observing the expression pattern of GRKs in the lesioned striatum after DA depletion and compared it with the levels in wt littermates. GRK3 and GRK6 which are down regulated in DA depleted striatum as seen in PD remained unchanged or slightly higher in the Hdc-KO mice striatum lesioned with 6-OHDA. Loss of GRK3 and GRK6 in the striatum of PD brains results in the supersensitive response of DA receptors upon DA replacement therapy with L-DOPA. The levels of GRK3 and GRK6 are not reversed by DOPA treatment in PD animal models. However, lentivirus mediated overexpression of GRK3 and GRK6 in the striatum of rat and monkey models of PD respectively resulted in significant down regulation of LID. It has been previously shown that both GRK3 and GRK6 target different signaling mechanisms to counteract dyskinesia. GRK6 predominantly acts through its kinase domain to phosphorylate the agonist occupied DA D1 receptors, whereas GRK3 acts via its RGS domain to counteract dyskinesia by sequestering G_{αq} signaling. Since the loss of GRK3 and GRK6 in the PD patient brains are permanent, attempts have been made to develop ideal drug candidates that could enhance the expression of these two rate limiting kinases in the brain. Down regulating the signaling of HA in the brain results in the upregulation of GRK3 and GRK6. Loss of DA does not appear to play a role in the down regulation of GRK3 and GRK6 in Hdc-KO mice, however a huge morbidity was observed (66% loss, n=12, with 8 dead during post-surgical recovery of two weeks) in these mice model when DA was depleted by 6-OHDA infusion in to the median forebrain bundle to destroy the dopaminergic projection neurons. The exact reason for the morbidity is not known since the wt littermates didn't show such a huge morbidity (n=12, with 1 dead post-surgery within two weeks of surgery). The expression pattern of GRKs and other significant signaling pathways in the remaining mouse (n=4 for Hdc-KO) was observed. The levels of GRK3 and GRK6 were unchanged in the lesioned striatum of the HDCko mouse lines suggesting the significant role played by HA downregulation in PD. The interaction of dual loss of DA and HA has not been previously reported before as far as the expression of GRKs are concerned. One would expect, loss of HA along with DA would further reduce the expression of GRKs, but the opposite effect was observed in Hdc-KO mouse lines. The exact mechanism by which the loss of HA results in the upregulation of GRKs is not known at this point and needs further investigation. The fact that loss of HA resulted in the upregulation of GRKs especially GRK3 and GRK6, which are highly impacted in the PD animal models was taken advantage of to develop therapeutic drug candidates. Previous studies using behavioral and electrophysiological approaches with H2R antagonists have shown beneficial outcomes in PD, but the molecular mechanism has not been known.

It has been suggested that H2R is selectively down regulated by GRK mediated phosphorylation especially by GRK2 and GRK3 and GRK5 and GRK6 seems to play little or no role in the desensitization of the H2R. The specificity of protein kinase mediated phosphorylation of H2R specifically by GRK2 and GRK3 suggests that GRK3 could be constitutively active when there is sensitization of the histaminergic system as in the case of loss of HA in Hdc-KO mice leading to over activation of H2R. This mechanism may be preserved in the knockout mouse line even when they are targeted for DA depletion with unilateral injection of 6-OHDA to destroy dopaminergic projection neurons. The histaminergic and dopaminergic system innervate the striatum, but their projection neurons originate at different locations of the brain and targeting with 6-OHDA only destroys the dopaminergic projection neurons from the substantia nigra and not histaminergic neurons from the tuberomammillary nucleus of the hypothalamus.

If GRK3 was upregulated when there is a loss of HA activity, blocking the histaminergic action by using receptor antagonist against H2R could have beneficial outcome in PD by enhancing the expression of GRK3. Suppressing the histaminergic input in to the striatum especially on to the MSNs and cholinergic system could provide tonic inhibition and reduce neurotransmitter release thereby impeding the firing potential, since abnormal striatal cholinergic tone has been reported to contribute to LID. Acetylcholine activates both nicotinic and muscarinic receptors in the striatum leading to LID and blocking these receptors counteracts LID. It has also been reported that enhanced histamine H2R excitation of the striatal cholinergic interneurons as a possible mechanism for LID in animal models of PD. Use of famotidine, a H2R antagonist effectively counteracted the hyperactive cholinergic interneurons and decreased the dyskinetic behavior following DOPA treatment. The dorsolateral cholinergic interneurons display a stronger excitatory response to DA resulting in enhanced H2R signaling leading to LID. Previous studies have reported that the behavioral expression of LID has been associated with increased phosphorylation of the extracellular signaling regulated kinase (ERK) in cholinergic interneurons. In another mouse model, the ablation of striatal cholinergic interneurons resulted in reduced dyskinetic behavior due to DOPA therapy. These previous studies have indicated the modulation of striatal cholinergic tone could be a potential target for treating LID. Ranitidine was used in the drug formulation which has been previously shown to suppress LID in animal models of PD to elucidate the mechanistic pathways of LID amelioration in hemiparkinsonian mice model. Focus was directed on the GRK pathway especially of GRK3 and GRK6 to counteract the effect of LID and check whether there were changes in the levels of GRKs with ranitidine treatment along with DOPA. Ranitidine treatment enhanced the expression of GRK3 and GRK6 in the lesioned striatum than the DOPA treatment group. Addition of ranitidine had a profound effect on the LID in the mouse model tested and it may be through the increased expression of GRK3 in the lesioned striatum since DOPA treatment didn't have any effect on the upregulation of GRK3. The signaling studies of the MAPK pathways suggests ranitidine when given alone significantly increased ERK activity in the lesioned hemisphere and it is possible this ERK activity was coming from in the striatal region. The ERK activation in control DOPA and formulation groups was qualitatively evaluated and it was observed that the activation was mostly centered in the ventromedial and ventrolateral striatum and there was fewer colocalization of pERK and ChAT positive neurons in the DOPA and formulation groups.

Increased ChAT immunoreactivity of CINs was found in the lesioned side of the hemiparkinsonian mice in the formulation groups which positively correlates with the reduced dyskinetic behavior seen with formulation group than DOPA control group animals. The expression of LID markers such as activated ERK and FosB in the ChAT positive CINs was then measured. The majority of the ChAT positive interneurons were either lacking pERK staining in the dyskinetic animals suggesting that the ERK activity was perhaps deactivated with chronic DOPA treatment in these animals as observed in previous signaling studies when the hemiparkinsonian rats were chronically treated with L-DOPA, they lose ERK activation. Many of the signaling pathways are deregulated in the striatum due to DA depletion and subsequent L-DOPA replacement therapy as in PD. The DA receptor sensitization has been thought to play a major role in the abnormal signaling seen which eventually leads to irreversible signaling plasticity. Although L-DOPA gives much of the clinical relief in PD the management is always complicated due to the development of the debilitating side effects and a clear understanding of the signaling pathways becomes vital. The ERK and the Δ Fos B are the best studied pathways in the context of DA depletion and subsequent L-DOPA therapy. Enhanced ERK response to dopaminergic stimulation in the DA depleted striatum has been linked to the D1 receptor supersensitivity, on the other hand Δ Fos B tends to accumulate in the DA depleted striatum with subsequent L-DOPA treatment and has been shown to contribute to the development of LID. The localization of ERK and Δ Fos B in the DA depleted striatum with subsequent L-DOPA therapy and with ranitidine/DOPA formulations was observed. There was minimal co-localization of active ERK and Δ Fos B in the striatum with the localization segregated in the striatum. ERK activation was more seen in the ventromedial and ventrolateral striatum next to nucleus accumbens with minor staining in the dorsomedial striatum. Δ Fos B accumulation was more prominent in the dorsomedial and dorsolateral regions in the DOPA treated striatum. Nevertheless subtle changes in the expression pattern of ERK and Δ Fos B with ranitidine has been beneficial in ameliorating the LID. Previous studies showed that the overexpression of GRK3 and RGS domain of GRK3 had been successful in dampening down LID by suppressing the accumulation of Δ Fos B in hemiparkinsonian rats. On the other hand down regulation of GRK3 by lentivirus mediated knockdown resulted in the excessive accumulation of Δ Fos B which could be reflected in the increased contralateral locomotor behavior and dyskinesia in rats. The role of ranitidine in reducing dyskinesia has been previously reported, however the exact signaling mechanism by which ranitidine counteracts dyskinesia has not been studied before. Even marginal cell specific increases in the levels of GRK3 could be beneficial in counteracting the signaling deficits due to DOPA therapy and ranitidine provides it in the DA depleted striatum. Taken together these results suggest the idea that H2R antagonism might be a beneficial therapeutic option in DA replacement therapy with L-DOPA in alleviating the debilitating side effects of L-DOPA in PD.

It is shown herein that the mechanistic pathway by which the H2R antagonist ranitidine inhibited the LID via normalizing the levels of GRK3 in the DA depleted striatum. Although GPCRs are phosphorylated in a number of ways, GRKs are the primary kinases that are responsible to effectively shut down of the physiological responses following cell stimulation. In diseased conditions such as Parkinson's there is significant loss of GRK3 and GRK6 in the dopamine depleted striatum. These changes tend to be irreversible even after DOPA treatment which is the current golden standard for the clinical manifestation of PD. In previous PD studies, it has been shown that lentivirus mediated overexpression of GRK3 and GRK6 in the DA depleted striatum in rat and monkey models respectively have shown beneficial effects of L-DOPA therapy. The same beneficial effects of DOPA seen in gene therapy using GRK3 and GRK6 via ranitidine/DOPA formulations targeting the histaminergic system are recapitulated. The results are substantiated using the PD model of Hdc-KO mouse line which has constitutive depletion of HA in the brain due to the loss of histidine decarboxylase the enzyme responsible for the conversion of histidine to histamine. DA depletion in this mouse line showed enhanced levels of GRK3 and GRK6 mimicking the effect seen with ranitidine/DOPA treatments in wt 6-OHDA lesioned mice. These results suggests that ranitidine ameliorates dyskinetic behavior seen in chronic DOPA treated animals by enhancing the levels of GRK3 by modulating the H2R activity.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A formulation, comprising L-DOPA, a decarboxylase inhibitor, and ranitidine dispersed in a polymer.

2. The formulation of claim 1, wherein the decarboxylase inhibitor is carbidopa, wherein the weight ratio of L-DOPA to carbidopa is in the range of about 1:(0.2-0.3).

3. The formulation of claim 2, wherein the weight ratio of L-DOPA to carbidopa is in the range of about 1:(0.24-0.26).

4. The formulation of claim 2, wherein the weight ratio of L-DOPA to carbidopa is in the range of about 1:0.25.

5. The formulation of claim 1, wherein the decarboxylase inhibitor is benserazide, wherein the weight ratio of L-DOPA to benserazide is in the range of about 1:(0.4-0.6).

6. The formulation of claim 5, wherein the weight ratio of L-DOPA to benserazide is in the range of about 1:(0.45-0.55).

7. The formulation of claim 5, wherein the weight ratio of L-DOPA to benserazide is in the range of about 1:0.5.

8. The formulation of claim 1, wherein the weight ratio of L-DOPA to ranitidine is the range of about 1:(0.75-1.25).

9. The formulation claim 1, wherein the polymer is selected from the group of gelatin, a polymer of acrylic acid, a polymer of methyl methacrylate, chitosan, pullulan, and combinations thereof.

10. The formulation of claim 1, wherein the polymer comprises gelatin type A.

11. The formulation of claim 1, wherein the weight ratio of L-DOPA to the polymer is in the range of about 1:(0.75-1.25).

12. The formulation of claim 1, wherein the formulation is in the form of a tablet or a capsule.

13. The formulation of claim 1, further comprising a pharmaceutically acceptable carrier.

14. The formulation of claim 1, wherein the decarboxylase inhibitor is carbidopa, and the polymer is a gelatin, wherein the weight ratio of L-DOPA:carbidopa:ranitidine:gelatin is about 1:0.25:1:1.

15. The formulation of claim 1, wherein the decarboxylase inhibitor is benserazide, and the polymer is a gelatin, wherein the weight ratio of L-DOPA:benserazide:ranitidine:gelatin is about 1:0.5:1:1.

16. A method of preventing or treating dyskinesia in a patient suffering from Parkinson's disease (PD), comprising administering to the patient an effective amount of a formulation comprising L-DOPA, a decarboxylase inhibitor, and ranitidine dispersed in a polymer.

17. The method of claim 16, wherein the patient suffers from dyskinesia induced by a prior treatment of L-DOPA.

18. The method of claim 16, wherein the decarboxylase inhibitor is carbidopa, wherein the weight ratio of L-DOPA to carbidopa is in the range of about 1:(0.2-0.3).

19. The method of claim 16, wherein the decarboxylase inhibitor is benserazide, wherein the weight ratio of L-DOPA to benserazide is in the range of about 1:(0.4-0.6).

20. The method of claim 16, wherein the L-DOPA and the decarboxylase inhibitor are administered in separate formulations or in a single formulation.
